(19) United States
(12) Patent Application Publication
(10) Pub. No.: US 2020/0340006 A1
(43) Pub. Date: Oct. 29, 2020

(54) RECOMBINANT WRKY POLYNUCLEOTIDES, WRKY MODIFIED PLANTS AND USES THEREOF

(71) Applicant: University of North Texas, Denton, TX (US)

(72) Inventors: JYOTI SHAH, Denton, TX (US); MONIKA ASHWINKUMAR PATEL, Lewisville, TX (US); VIJEE MOHAN, Denton, TX (US)

(21) Appl. No.: 16/831,138

(22) Filed: Mar. 26, 2020

Related U.S. Application Data
(60) Provisional application No. 62/823,900, filed on Mar. 26, 2019.

(51) Int. Cl. C12N 15/82; C07K 14/415

(52) U.S. Cl. CPC C12N 15/8273; C07K 14/415; C12N 15/8286

(57) ABSTRACT

Described herein are recombinant polynucleotides and vectors that can encode and/or express WRKY transcription factor polypeptides. Also described herein are recombinant WRKY transcription factor polypeptides. Also described herein are transgenic plant cells and plants that can overexpress a WRKY transcription factor polypeptide and methods of increasing tolerance to abiotic and/or biotic stressors in plants.

### CROSS-REFERENCE TO RELATED APPLICATIONS

This application claims the benefit of and priority to U.S. Provisional Application Ser. No. 62/823,900, having the title “WRKY MODIFIED PLANTS AND USES THEREOF”, filed on Mar. 26, 2019, which is herein incorporated by reference in its entirety.

### SEQUENCE LISTING

This application contains a sequence listing filed in electronic form as an ASCII.txt file entitled 921402-1030_ST25.K created on Mar. 26, 2020 and having a size of 26 KB. The content of the sequence listing is incorporated herein in its entirety.

### BACKGROUND

Plants are continuously challenged by abiotic (e.g. drought and salt) and biotic (e.g.

insects) stresses. Infestation by the green peach aphid (Myzus persicae), is generally recognized as one of the most common damaging insect pests to several species of plants, including agricultural and horticultural plants in over 50 plant families. Drought and salinity also have a strong impact on agricultural economics. For example, recent droughts in the US caused billions in agricultural losses, and salt stress on the world's agricultural land leads to significant losses in yield and profit each year. As such, there exists a need to improve plant production particularly in geographical locations or temporal periods suffering from abiotic and biotic stressors.

### SUMMARY

Briefly described, in various aspects, the present disclosure provides isolated and recombinant polynucleotides encoding a WRKY peptide, isolated WRKY peptides, vectors including recombinant polynucleotides encoding WRKY peptides, cells and plants transformed with recombinant WRKY polynucleotides, and methods of increasing tolerance to abiotic and biotic stressors in plants.

The present disclosure provides recombinant polynucleotides of the present disclosure encoding a WRKY polypeptide. In embodiments, the recombinant polynucleotides can include a WRKY45 polynucleotide having a sequence that is about 50-100% identical to any one of SEQ ID NOs: 1-3, and at least one heterologous polynucleotide sequence operatively linked to the WRKY45 polynucleotide. The heterologous polynucleotide can be, but is not limited to, a regulatory polynucleotide sequence, a selectable marker polynucleotide, or combinations of both. Embodiments of the present disclosure also include vectors including the recombinant polynucleotide of the disclosure.

The present disclosure also provides cells including a recombinant polynucleotide or vector of the present disclosure. The cells can be plant, bacteria, yeast, or fungal cells.

Embodiments of the present disclosure also include transgenic plants grown from the cells of the present disclosure including the recombinant polynucleotide or vector of the present disclosure. In embodiments, a transgenic plant of the present disclosure can include a plurality of cells, where one or more of the plurality of cells includes a recombinant polynucleotide or vector of the present disclosure. In embodiments transgenic plants of the present disclosure express an increased amount of a WRKY transcription factor protein as compared to a non-transgenic control plant. In embodiments, the transgenic plants of the present disclosure have increased tolerance to biotic and/or abiotic stressors.

Methods of the present disclosure include methods of increasing tolerance to an abiotic or biotic stressor in a plant. In embodiments, methods of the present disclosure include integrating into the genome of at least one cell of a plant a recombinant polynucleotide of or a vector of the present disclosure, such that the recombinant polynucleotide is expressed in the plant cell; and growing said plant, wherein the recombinant polynucleotide is overexpressed in the plant relative to a wild-type plant and wherein the plant has increased tolerance, as compared to a non-transgenic control plant or wild type plant, to one or more abiotic stressors, one or more biotic stressors, or a combination thereof.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a phylogeny diagram for WRKY45.

FIG. 2 is a graph illustrating AtWRKY45 expression in Arabidopsis in response to infestation with green peach aphid (GPA).

FIGS. 3A-3G are images visually illustrating AtWRKY45 promoter activity with GUS expression to show the location of AtWRKY45 expression in the plant in response to GPA infestation.

FIGS. 4A-4B illustrate overexpression of AtWRKY45 (FIG. 4A) and the result of overexpression on number of GPA progeny per plant per day as compared to a wild type plant (FIG. 4B).

FIGS. 5A-5B illustrate the effect of AtWRKY45 overexpression on draught tolerance in Arabidopsis. FIG. 5A provides images of wild type vs. AtWRKY45 overexpression plants under watered conditions, drought conditions, and post drought/re-watered conditions. FIG. 5B is a graph comparing survival rate of wild type plants vs AtWRKY45 overexpression plants after recovery from drought.

FIG. 6 is a graph illustrating the effect of AtWRKY45 overexpression in Arabidopsis on stomatal closure during drought stress as monitored by stomatal aperture size measurements.

FIG. 7 is a graph showing that AtWRKY45 overexpression in Arabidopsis enhances stomatal closure during drought stress as measured by thermal imaging.

FIGS. 8A-8B illustrate the effect of AtWRKY45 overexpression on salt tolerance in Arabidopsis. FIG. 8A provides images of wild type vs. AtWRKY45 overexpression plants after exposure to salt water vs. normal water. FIG. 8B is a graph comparing survival rate of wild type plants vs AtWRKY45 overexpression plants after recovery salt exposure.

FIG. 9 is a graph demonstrating that AtWRKY45 overexpression in Arabidopsis results in constitutively higher expression of genes associate with stress response.

FIG. 10 illustrates elevated levels of the plant stress-adaptation hormone ABA in Arabidopsis as a result of AtWRKY45 overexpression.

FIGS. 11A-11F are a series of graphs illustrating elevated expression of stress associated genes in Arabidopsis in response to AtWRKY45 overexpression. Overexpressed genes, in addition to WRKY45 (FIG. 11A), include: KIN1 (FIG. 11B), COR47 (FIG. 11C), DREB2A (FIG. 11D), NCED3 (FIG. 11E), and RD29A (FIG. 11F).

FIG. 12 is a graph illustrating expression of A. thaliana WRKY45 CDS (SEQ ID NO: 3) in tomato (Solanum lycopersicum) and effect on resistance to green peach aphid (Myzus persicae) compared to a non-transgenic tomato cultivar.

FIGS. 13A-13B illustrate the effect of expression of A. thaliana WRKY45 CDS (SEQ ID NO: 3) in tomato (Solanum lycopersicum) on drought tolerance/recovery. FIG. 13A is a digital image illustrating three AtWRKY45 transgenic lines and one non-transgenic line, and FIG. 13B is a bar graph illustrating plant survival after drought exposure.

FIGS. 14A-14B illustrate the effect of expression of A. thaliana WRKY45 CDS (SEQ ID NO: 3) in tomato (Solanum lycopersicum) on salt stress. FIG. 14A is a digital image illustrating a AtWRKY45 transgenic line compared to a non-transgenic line, and FIG. 14B is a bar graph illustrating plant growth under salt stress of 2 transgenic AtWRKY45 lines as compared to non-transgenic lines.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used in the specification and the appended claims, the singular forms “a,” “an,” and “the” include plural referents unless the context clearly dictates otherwise.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Embodiments of the present disclosure will employ, unless otherwise indicated, techniques of molecular biology, microbiology, genetic engineering, organic chemistry, biochemistry, physiology, cell biology, plant physiology, plant pathology, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible unless the context clearly dictates otherwise.

### Definitions

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein with reference to the relationship between DNA, cDNA, mRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer

RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “DNA molecule” can include nucleic acids/polynucleotides that are made of DNA.

As used herein, the term “encode” refers to principle that DNA can be transcribed into RNA, which can then be translated into amino acid sequences that can form proteins.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “identity,” can refer to a relationship between two or more nucleotide or polypeptide sequences, as determined by comparing the sequences. In the art, “identity” can also refer to the degree of sequence relatedness between nucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure, unless stated otherwise.

As used herein, “nucleic acid,” “nucleotide sequence,” and “polynucleotide” can be used interchangeably herein and can generally refer to a string of at least two base-sugar-phosphate combinations and refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids.

Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “operatively linked” in the context of recombinant DNA molecules, vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms also contemplate plants, fungi, bacteria, etc.

As used herein, “overexpressed” or “overexpression” refers to an increased expression level of an RNA and/or protein product encoded by a gene as compared to the level of expression of the RNA or protein product in a normal or control cell. The amount of increased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 0, 90, 100 fold or more greater than the normal or control cell.

As used herein, the terms “optional” or “optionally” means that the subsequently described event or circumstance can or cannot occur, and that the description includes instances where said event or circumstance occurs and instances where it does not.

As used herein, “plasmid” refers to a non-chromosomal double-stranded DNA sequence including an intact “replicon” such that the plasmid is replicated in a host cell.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to heterologous sequence (e.g., a regulatory sequence such as, but not limited to, a promoter sequence, where the coding sequence and heterologous sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “selectable marker” refers to a gene whose expression allows one to identify cells that have been transformed or transfected with a vector containing the marker gene. For instance, a recombinant nucleic acid may include a selectable marker operatively linked to a gene of interest and a promoter, such that expression of the selectable marker indicates the successful transformation of the cell with the gene of interest.

A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “transforming” when used in the context of engineering or modifying a cell, refers to the introduction by any suitable technique and/or the transient or stable incorporation and/or expression of an exogenous gene in a cell. It can be used interchangeably in some contexts herein with “transfection”.

As used herein, the term “transfection” refers to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA, it may be associated with various proteins or regulatory elements (e.g., a promoter and/or signal element), or the nucleic acid may be incorporated into a vector or a chromosome.

As used herein, “variant” can refer to a polynucleotide or polypeptide that differs from a reference polynucleotide or polypeptide but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, the term “vector” is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

As used herein, “wild-type” is the typical form of an organism, variety, strain, gene, protein, or characteristic as it occurs in nature, as distinguished from mutant forms that may result from selective breeding or transformation with a transgene.

As used herein, “electroporation” is a transformation method in which a high concentration of plasmid DNA (containing exogenous DNA) is added to a suspension of host cell protoplasts, and the mixture shocked with an electrical field of about 200 to 600 V/cm.

As used herein, a “transgene” refers to an artificial gene which is used to transform a cell of an organism, such as a bacterium or a plant.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

### Discussion

Plants are continuously challenged by abiotic (e.g. drought and salt) and biotic (e.g. insects) stresses. WRKY family of transcription factors (TFs) are one of the largest families of regulatory proteins in plants that have been shown to play important roles in responses to biotic and abiotic stresses. In addition to higher plants (rice, soybean, Arabidopsis, tobacco), WRKY TFs have also been identified in protists like Giardia lamblia and Dictyostelium discoideum, unicellular green algae like Chlamydomonas reinhardtii, multicellular green algae like Kiebsormidium flaccidum, and mosses like Physcomitrella patens. Presence of WRKY genes in lower life forms indicates an ancient origin of this gene family.

Arabidopsis thaliana, a relative of plants in the Crucifer family, which includes cauliflower, cabbage, turnip, mustard, and canola, contains over 70 WRKY genes. The WRKY45 (At3g01970) gene in Arabidopsis thaliana was shown to be involved in phosphate starvation and age-triggered senescence. The present disclosure, including the examples below, demonstrate that WRKY45 is involved in controlling infestation by the green peach aphid (Myzus persicae), which is recognized as the 3rd most damaging insect pest of plants, including agricultural and horticultural plants in over 50 plant families. Increased WRKY45 expression can result in curtailment of aphid infestation. In addition, WRKY45 overexpression can confer enhanced tolerance to drought and salt stress. WRKY45 overexpression also promotes recovery from drought when plants are re-watered. Drought and salinity are two major abiotic stressors of plants that impact agricultural economics. For example, the 1998 and 2012 drought in the US caused agricultural losses to the extent of $40 billion. Similarly, about 20% of the world's agricultural land is affected by salt stress each year which leads to enormous amount of yield loss. As such, there exists a need to improve plant production particularly in geographical locations or temporal periods suffering from abiotic and biotic stressors.

The present disclosure thus provides compositions, plants and methods involving expression of recombinant nucleotides encoding WRK45 proteins and/or derivatives thereof. Embodiments of the present disclosure include at least recombinant polynucleotides that encode a WRKY polypeptide, vectors including the recombinant polynucleotides for expression of WRKY polypeptides, and genetically modified plants that can overexpress one or more WRKY proteins. Also described herein are methods of making and using that genetically modified plants that can overexpress one or more WRKY proteins, including methods of increasing tolerance of a plant to an abiotic or biotic stressor by genetically modifying the plant to express a recombinant polynucleotide encoding a WRKY protein. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Nucleic Acid Sequences

Isolated Nucleotide and cDNA Sequences

The present disclosure describes isolated nucleotide and cDNA sequences, which either in whole or in part, can encode a WRKY transcription factor protein. In some embodiments, the WRKY transcription factor protein encoded by an isolated or synthetic nucleotide or cDNA sequence can result in an improvement in plant response to an abiotic and/or biotic stressor.

In some embodiments, a nucleotide encoding a WRKY transcription factor protein can have an isolated nucleotide sequence according to or include any one of SEQ ID NOs: 1-3. In some embodiments, a cDNA corresponding to a WRKY transcription factor protein can have a sequence corresponding to any one of or include any one of SEQ ID NOs: 2-3. The isolated nucleotide and/or cDNA can have or include a sequence with about 50, 51, 52, 53, 54, 55, 56, 57, 58, 59, 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 1-3. The isolated nucleotide and/or cDNA can have or include a sequence with about 50, 51, 52, 53, 54, 55, 56, 57, 58, 59, 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 2-3. In some embodiments, a WRKY transcription factor protein cDNA encodes a polypeptide having a sequence about 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 4-16. Suitable nucleotide sequences can be obtained by using standard methods known to those of skill in the art, including but not limited to, restriction enzyme digestion and polymerase chain reaction (PCR), or de novo nucleotide sequence synthesis techniques.

Recombinant Polynucleotide Sequences

The present disclosure also includes recombinant polynucleotide sequences having any of the isolated nucleotide or cDNA sequences or fragments thereof previously described and at least one additional heterologous polynucleotide sequence operatively linked to the isolated nucleotide or cDNA sequences or fragments thereof. In embodiments recombinant polynucleotides of the present disclosure encode a WRKY45 polynucleotide having a sequence that is about 50-100% identical to any one of SEQ ID NOs: 1-3 (as described above) and at least one heterologous polynucleotide sequence operatively linked to the WRKY45 polynucleotide. In some embodiments, heterologous polynucleotide sequences can include non-coding nucleotides that can be placed at the 5′ and/or 3′ end of the polynucleotides encoding a WRKY transcription factor protein without affecting the functional properties of the molecule. A polyadenylation region at the 3′-end of the coding region of a polynucleotide can be included. The polyadenylation region can be derived from the endogenous gene, from a variety of other plant genes, from T-DNA, or through chemical synthesis. In further embodiments, the nucleotides encoding the WRKY transcription factor protein may be conjugated to a nucleic acid encoding a signal or transit (or leader) sequence at the N-terminal end (for example) of the WRKY transcription factor protein that co-translationally or post-translationally directs transfer of the WRKY transcription factor protein. The polynucleotide sequence may also be altered so that the encoded root WRKY transcription factor protein is conjugated to a linker, selectable marker, or other sequence for ease of synthesis, purification, and/or identification of the protein. In embodiments, the recombinant polynucleotide sequence includes at least one regulatory sequence operatively linked to the isolated nucleotide or cDNA sequences or fragments thereof. In some embodiments the at least one regulatory sequence can include a promoter or other regulatory sequence to direct translation/expression of the encoded polypeptide.

To express an exogenous WRKY transcription factor protein gene, fragment thereof, or antisense nucleotide in a cell, in embodiments, the exogenous nucleotide can be combined (e.g., in a vector) with transcriptional and/or translational initiation regulatory sequences, i.e. promoters, that direct the transcription of the gene and/or translation of the encoded protein in a cell. In some embodiments, a constitutive promoter may be employed. Suitable constitutive promoters for plant cells include, but are not limited to, the cauliflower mosaic virus (CaMV) 35S transcription initiation region, the 1′- or 2′-promoter derived from T-DNA of Agrobacterium tumefaciens, the ACT11 and Cat3 promoters from Arabidopsis (Huang et al. Plant Mol. Biol. 1996, 33:125-139 and Zhong et al. Mol. Gen. Genet. 1996, 251:196-203), the stearoyl-acyl carrier protein desaturase gene promoter from Brassica napus (Solocombe et al. Plant Physiol. 1994, 104:1167-1176), and the GPc1 and Gpc2 promoters from maize (Martinez et al. J. Mol. Biol. 1989, 208:551-565 and Manjunath et al. Plant Mol. Biol. 1997, 33:97-112). Suitable constitutive promoters for bacterial cells, yeast cells, fungal cells are generally known in the art, such as a T-7 promoter for bacterial expression and an alcohol dehydrogenase promoter for expression in yeast.

In other embodiments, tissue-specific promoters or inducible promoters may be employed to direct expression of the exogenous nucleic acid in a specific cell type, under certain environmental conditions, and/or during a specific state of development. In some embodiments, the tissue-specific promoter can be a root-specific or a phloem-specific promoter. Suitable root specific and phloem-specific promoters are generally known in the art. Examples of environmental conditions that may affect transcription by inducible promoters include anaerobic conditions, elevated temperature, the presence of light, contact with chemicals or hormones, or infection by a pathogen. Suitable plant inducible promoters include the root-specific ANRI promoter (Zhang and Forde. Science. 1998, 279:407), the photosynthetic organ-specific RBCS promoter (Khoudi et al. Gene. 1997, 197:343), the tomato fruit ripening-specific E8 promoter (Deikman, J., et al. Plant Physiol. 1992, 100: 2013-2017), the salicylic acid-inducible PR1 promoter (Lebel et al. Plant Journal. 1998, 16:223-233), and the phloem specific SUC2 promoter.

A selectable marker can also be included in the recombinant nucleic acid to confer a selectable phenotype on plant cells. For example, the selectable marker may encode a protein that confers biocide resistance, antibiotic resistance (e.g., resistance to kanamycin, G418, bleomycin, hygromycin), or herbicide resistance (e.g., resistance to chlorosulfuron or Basta). Thus, the presence of the selectable phenotype indicates the successful transformation of the host cell. An exemplary selectable marker includes the beta-glucuronidase (GUS) reporter gene.

Suitable recombinant polynucleotides can be obtained by using standard methods known to those of skill in the art, including but not limited to, restriction enzyme digestion, PCR, ligation, and cloning techniques.

### Isolated Protein (Polypeptide) and Peptide Sequences:

The present disclosure also describes an isolated or synthetic protein (polypeptide) corresponding to a WRKY transcription factor protein. In some embodiments, the isolated polypeptide has an amino acid sequence corresponding to any one of SEQ ID NOs: 4-16. In some embodiments, a WRKY transcription factor protein has a sequence at least 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 4-16.

Modifications and changes can be made in the structure of the polypeptides of the present disclosure that result in a molecule having similar characteristics as the unmodified polypeptide (e.g., a conservative amino acid substitution). Modification techniques are generally known in the art. For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a functional variant. Polypeptides with amino acid sequence substitutes that still retain properties substantially similar to or better than polypeptides corresponding to a WRKY transcription factor protein are within the scope of this disclosure. In some embodiments, the exogenous WRKY transcription factor protein can have enhanced activity as compared to wild-type.

The present disclosure also includes isolated and synthetic peptides corresponding to a fragment of the polypeptide corresponding to a WRKY transcription factor protein. In some embodiments the peptides correspond to a portion of any one of SEQ ID NOs: 4-16. The isolated or synthetic peptides have about 80, 81, 82, 83, 84, 85, 86, 87, 88, 89, 90, 91, 92, 93, 94, 95, 96, 97, 98, 99 to 100% identity to a portion of any one of SEQ ID NOs: 4-16 that are at least 10 amino acids long.

In other embodiments, the isolated or synthetic peptide as described herein is suitable for use in production of antibodies against a WRKY transcription factor protein. In other words, the isolated or synthetic peptide as described herein serves as the antigen to which an antibody is raised against. In some embodiments, the isolated or synthetic peptide sequence is also the epitope of the antibody. Antibodies raised against a WRKY transcription factor protein are suitable for use in methods for at least detection, quantification, and purification of a WRKY transcription factor protein. Other uses for anti-WRKY transcription factor protein antibodies are generally known in the art.

### Vectors

Vectors having one or more of the polynucleotides or antisense polynucleotides described herein can be useful in producing transgenic bacterial, fungal, yeast, plant cells, and transgenic plants that express varying levels of a WRKY transcription factor protein. Within the scope of this disclosure are vectors containing one or more of the polynucleotide sequences described herein.

In embodiments, the vector includes a polynucleotide encoding a WRKY transcription factor protein, where the polynucleotide has about 50, 51, 52, 53, 54, 55, 56, 57, 58, 59, 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 1-3. In embodiments, the vector includes a polynucleotide that can encode a WRKY transcription factor protein, wherein the protein has about 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 4-16. In embodiments the vector includes the recombinant polynucleotide of the present disclosure including a WRKY45 polynucleotide having a sequence that is about 50-100% identical to any one of SEQ ID NOs: 1-3, and at least one heterologous polynucleotide sequence operatively linked to the WRKY45 polynucleotide.

In embodiments, the vector has at least one regulatory sequence operatively linked to a DNA molecule or encoding a WRKY transcription factor protein such that the WRKY transcription factor protein is expressed in a bacteria, fungus, yeast, plant, or other cell into which it is transformed. In other embodiments, the vector includes a promoter that serves to initiate expression of the WRKY transcription factor protein such that the WRKY transcription factor protein is over-expressed in a plant cell into which it is transformed relative to a wild-type bacteria, fungus, yeast, or plant cell. In some embodiments, the vector has at least one regulatory sequence operatively linked to a DNA molecule encoding a WRKY transcription factor protein and a selectable marker.

Other embodiments of the present disclosure include a vector having an antisense polynucleotide capable of inhibiting expression of an endogenous the WRKY transcription factor protein gene and at least one regulatory sequence operatively linked to the antisense polynucleotide such that the antisense polynucleotide is transcribed in a type bacteria, fungus, yeast, or plant cell into which it is transfected. In embodiments, the antisense polynucleotides may be capable of inhibiting expression of an endogenous WRKY transcription factor protein gene corresponding to or including any one of SEQ ID NOs: 1-3 or about 50, 51, 52, 53, 54, 55, 56, 57, 58, 59, 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 1-3.

### Transgenic Cells, Organisms, and Plants

The polynucleotide sequences and vectors described above can be used to transform cells (e.g., plant cells) and to produce transgenic plants. The present disclosure provides transformed cells including the recombinant polynucleotides and/or vectors of the present disclosure described above including a WRKY45 polynucleotide having a sequence that is about 50-100% identical to any one of SEQ ID NOs: 1-3, and at least one heterologous polynucleotide sequence operatively linked to the WRKY45 polynucleotide. In embodiments the heterologous polynucleotide sequence includes a regulatory polynucleotide sequence, a selectable marker polynucleotide, or both. In embodiments the cell can be a plant cell, bacterial cell, yeast cell, of fungus cell. Also, within the scope of this disclosure are populations of cells where about 1% to about 100%, or between about 50% and about 75%, or between about 75% and about 100% of the cells within the population contain a vector as previously described. In some embodiments, the cell is a plant cell, such as, but not limited to: Arabidopsis, rice, wheat, barley, cotton, rose, china rose, apple, camelina, peach, maize, tobacco, soybean, Brassicas, tomato, potato, bell pepper, alfalfa, chickpea, sugarcane, sorghum, eggplant, sweet pepper, papaya, tobacco, cannabis, and/or canola cell.

In some embodiments, one or more cells within the population contain more than one type of vector. In some embodiments, all (about 100%) the cells that contain a vector have the same type of vector. In other embodiments, not all the cells that contain a vector have the same type of vector or plurality of vectors. In some embodiments, about 1% to about 100%, or between about 50% and about75%, or between about 75% and about 100% of the cells within the population contain the same vector or plurality of vectors. In some cell populations, all the cells are from the same species. Other cell populations contain cells from different species. Transfection methods for establishing transformed (transgenic) cells are well known in the art

In addition, the present disclosure provides transgenic organisms produced/grown from the transformed cells of the present disclosure. The present disclosure includes transgenic plants having a plurality of cells where one or more cells of the plurality of cells contain any of the recombinant polynucleotides or vectors previously described that have DNA sequences encoding the WRKY transcription factor protein. In one embodiment, the recombinant polynucleotide contains at least one regulatory element operatively linked to a WRKY transcription factor protein polynucleotide sequence about 50, 51, 52, 53, 54, 55, 56, 57, 58, 59, 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 1-3.

Also described herein are transgenic plants having one or more cells transformed with vectors containing any of the nucleotide sequences described above, and/or fragments of the nucleic acids encoding the WRKY transcription factor protein(s) of the present disclosure. In some embodiments, the vector contains a WRKY transcription factor protein polynucleotide having about 50, 51, 52, 53, 54, 55, 56, 57, 58, 59, 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 1-3.The transgenic plant can be made from any suitable plant species or variety including, but not limited to Arabidopsis, rice, wheat, barley, cotton, rose, china rose, apple, camelina, peach, maize, tobacco, soybean, Brassicas, tomato, potato, bell pepper, alfalfa, chickpea, sugarcane, sorghum, eggplant, sweet pepper, papaya, tobacco, cannabis, and/or canola. In embodiments, transgenic plants of the present disclosure include Arabidopsis and tomato plants.

In some embodiments, the transgenic plant having a nucleotide sequence encoding a WRKY transcription factor protein has increased expression of the a WRKY transcription factor protein relative to a wild type plant/non-transgenic control. In other embodiments, the transgenic plant having a nucleotide sequence encoding a WRKY transcription factor protein has increased expression of a WRKY transcription factor protein relative to a wild type plant and produces a WRKY transcription factor protein. The transgenic plant can have increased tolerance to a biotic and/or abiotic stressor. Abiotic stressors include but are not limited to, drought, salinity, heat, cold, pH, high light, ultra-violet light, and/or ozone. The transgenic plant can have increased tolerance to a biotic stressor. Biotic stressors can include insect and nematode infestation, bacterial infection, fungal infection, oomycete infection, mycoplasma infection and/or viral infection. In embodiments the transgenic plants of the present disclosure have increase tolerance to abiotic stressors such as, but not limited to drought and/or salinity. In embodiments, transgenic plants of the present disclosure can have increase tolerance to biotic stressors such as, but not limited to insects, including but not limited, to green peach aphid (Myzus persicae). Transgenic plants of the present disclosure can have increased tolerance to two or more biotic and/or abiotic stressors. For instance, transgenic plants of the present disclosure can have increased tolerance to salinity, drought, and certain insects.

A transformed plant cell of the present disclosure can be produced by introducing into a plant cell one or more vectors as previously described. In one embodiment, transgenic plants of the present disclosure can be grown from a transgenic plant cell transformed with one or more of the vectors previously described. In one embodiment, the cells are transformed with a vector including a recombinant polynucleotide encoding a WRKY transcription factor protein having about 50, 51, 52, 53, 54, 55, 56, 57, 58, 59, 60, 61, 62, 63, 64, 65, 66, 67, 68, 69, 70, 71, 72, 73, 74, 5, 76, 77, 78, 79, 80, 81, 82, 83, 84, 85, 86, 87, 88, 89, 90, 91, 92, 93, 94, 95, 96, 97, 98, 99 to 100% identity to any one of SEQ ID NOs: 1-3 that has at least one regulatory sequence operatively linked to the recombinant polynucleotide.

Techniques for transforming a wide variety of plant cells with vectors or naked nucleic acids are well known in the art and described in the technical and scientific literature. See, for example, Weising et al. Ann. Rev. Genet. 1988, 22:421-477. For example, the vector or naked nucleic acid may be introduced directly into the genomic DNA of a plant cell using techniques such as, but not limited to, electroporation and microinjection of plant cell protoplasts, or the recombinant nucleic acid can be introduced directly to plant tissue using ballistic methods, such as DNA particle bombardment.

Microinjection techniques are known in the art and well described in the scientific and patent literature. The introduction of a recombinant nucleic acid using polyethylene glycol precipitation is described in Paszkowski et al. EMBO J. 1984, 3:2717-2722. Electroporation techniques are described in Fromm et al. Proc. Natl. Acad. Sci. USA. 1985, 82:5824. Ballistic transformation techniques are described in Klein et al. Nature. 1987, 327:70-73. The recombinant nucleic acid may also be combined with suitable T-DNA flanking regions and introduced into a conventional Agrobacterium tumefaciens host vector, or other suitable vector. The virulence functions of the Agrobacterium tumefaciens host will direct the insertion of the recombinant nucleic acid including the exogenous nucleic acid and adjacent marker into the plant cell DNA when the cell is infected by the bacteria. Agrobacterium tumefaciens-mediated transformation techniques, including disarming and use of binary vectors, are known to those of skill in the art and are well described in the scientific literature. See, for example, Horsch et al. Science. 1984, 233:496-498; Fraley et al. Proc. Natl. Acad. Sci. USA. 1983, 80:4803; and Gene Transfer to Plants, Potrykus, ed., Springer-Verlag, Berlin, 1995.

A further method for introduction of the vector or recombinant nucleic acid into a plant cell is by transformation of plant cell protoplasts (stable or transient). Plant protoplasts are enclosed only by a plasma membrane and will therefore more readily take up macromolecules like exogenous DNA. These engineered protoplasts can be capable of regenerating whole plants. Suitable methods for introducing exogenous DNA into plant cell protoplasts include electroporation and polyethylene glycol (PEG) transformation. Following electroporation, transformed cells are identified by growth on appropriate medium containing a selective agent.

The presence and copy number of the exogenous nucleic acid in a transgenic plant can be determined using methods well known in the art, e.g., Southern blotting analysis. Expression of an exogenous WRKY transcription factor protein in a transgenic plant may be confirmed by detecting an increase or decrease of mRNA or the WRKY transcription factor protein in the transgenic plant. Methods for detecting and quantifying mRNA or proteins are well known in the art.

Transformed plant cells that are derived by any of the above transformation techniques, or other techniques now known or later developed, can be cultured to regenerate a whole plant. In embodiments, such regeneration techniques may rely on manipulation of certain phytohormones in a tissue culture growth medium, typically relying on a biocide or herbicide selectable marker that has been introduced together with the exogenous nucleic acid. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. Plant Phys. 1987, 38:467-486.

Once the exogenous a WRKY transcription factor protein polynucleotide has been confirmed to be stably incorporated in the genome of a transgenic plant, it can be introduced into other plants by sexual crossing. Any of a number of standard breeding techniques can be used, depending upon the species to be crossed.

Methods of Increasing Tolerance to Abiotic and/or Biotic Stressors in a Plant

This disclosure also encompasses methods of increasing the tolerance to one or more abiotic or biotic stressors in a plant. Such stressors can include the abiotic and biotic stressors discussed above. In embodiments, methods of increasing the tolerance of a plant to one or more abiotic and/or biotic stressors include integrating into the genome of at least one cell of a plant a recombinant polynucleotide or a vector of the present disclosure previously described such that the recombinant polynucleotide is expressed in the plant cell. The method further includes growing the plant, where the recombinant polynucleotide is overexpressed in the plant relative to a wild-type plant and the plant has increased tolerance (as compared to a non-transgenic control plant or wild type plant) to one or more abiotic stressors, one or more biotic stressors, or a combination of abiotic and biotic stressors. The abiotic stressors include those discussed above, including, but not limited to, drought salinity, and the like. The biotic stressors include those discussed above, including, but not limited to, insects, bacterial infections, fungal infections, and the like. Methods of the present disclosure can increase the tolerance of a plant to a combination of two or more biotic and/or abiotic stressors. In embodiments, the plant can be, but is not limited to, Arabidopsis, rice, wheat, barley, cotton, rose, china rose, apple, camelina, peach, maize, tobacco, soybean, Brassicas, tomato, potato, bell pepper, alfalfa, chickpea, sugarcane, sorghum, eggplant, sweet pepper, papaya, tobacco, cannabis, and canola.

Additional details regarding the methods, and compositions of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

| SEQUENCES | |
|---|---|
| SEQ ID NO: 1 Arabidopsis thaliana Genomic WRKY45 (NCBI Gene ID: 821270) | |
| > NM_111063 327412-325952 Arabidopsis thaliana chromosome 3 sequence | |
| GTTTGAAATTTGAATCCATTGAACCAAAATTTGAAGGAGTTGCATATATAATAATATAAATCAGAATGAT | |
| GTAGCCGCCACACCTTTTTGTTTCCACAAAACTCTTTTTCTGTGATGGATCCGCTAATGTAGCCATATTT | |
| TCAATATATATCACTTTCTCTGGCATCTTCGCTACCGTGTACGTCTCTCTTTCTCTCCCTCCCCTCCTTG | |
| GCTTTTTTCAAGTTCCCACCATAAACGCAGAGGGAGTTAAGAAATGGAGGATAGGAGGTGTGATGTGTTG | |
| TTTCCATGTTCATCATCGGTTGATCCTCGCTTGACAGAGTTTCATGGGGTCGACAACTCTGCTCAGCCGA | |
| CAACATCATCCGAAGAGAAGCCAAGGAGTAAGAAGAAGAAGAAAGAGAGAGAAGCGAGGTACGCGTTCCA | |
| GACAAGAAGCCAGGTTGATATACTGGATGATGGATACAGGTGGAGGAAGTACGGCCAAAAAGCAGTCAAG | |
| AACAATCCATTCCCCAGGTACGTACTTAATCATCAATGAATGATACTGTATGCTTGCTATGGTGATCAAA | |
| TAAGAGACTCATATACATACATGTAAAATATGAATATATATATATATAGGAGCTATTATAAGTGCACAGA | |
| AGAAGGATGCAGAGTGAAGAAGCAAGTGCAGAGGCAATGGGGAGACGAAGGAGTGGTGGTGACGACATAC | |
| CAAGGTGTTCATACACATGCCGTTGATAAACCCTCTGATAATTTCCACCACATCTTGACACAAATGCACA | |
| TCTTCCCTCCCTTTTGCTTGAAGGAATGATTAGAGGAATTGGATTGTAATATTTACTTTCCCAAAAACGT | |
| TGGGCTCACACCATCAGACCTTTACTTTTAAACTAGCAGCAACTCACATATCTCAAAAATACTAATCCTT | |
| ATCTTTGTCTTTATGGGACCTTTGAATCCATCTGCTTTGGTGTCTTAGTCTCGGCTGCCCTGTAATCGAA | |
| AGTATATTCATCATCAAATTACCAAACATAAAGAAGCAATGATGAGTCTATCATCTACAAAAACAATGTT | |
| ATGTATCCCAAACCTACCGATTATTCCAAAACTAGTGACAAGCTAAGGATATTGTGGAGATGAAGATGAG | |
| AAAGAGTACGAAAGCTAACTTTGAGGTTTCTTCTTGGATCCAATTGCGAATATGCTTCACGTTTCGCTTT | |
| AGAACGGAGGACGCTTTCTTTGTTAGGCCCATTAGCCTGGGCTCTCGTGTTTTTCATAATGTCAAGTCAG | |
| CCCAACAAGCCCAAATCTTTACAAAAAGAACCAAGGACCATGTCATCCGGAATATGGTGATATTATTGGA | |
| TTATACCATTGGACCATTTAACACAAAAGCAAATATGCAACAGAATATATATAGATCACATAACAGTCAG | |
| AATCTTCTCAAAAGATATGCTTTCTTCCTTTTCTATTCTAGAGTGTTTTGATGACATCGGC | |
| SEQ ID NO: 2 AtWRKY45 cDNA sequence NM_111063 | |
| GTTTGAAATTTGAATCCATTGAACCAAAATTTGAAGGAGTTGCATATATAATAATATAAATCAGAATGAT | |
| GTAGCCGCCACACCTTTTTGTTTCCACAAAACTCTTTTTCTGTGATGGATCCGCTAATGTAGCCATATTT | |
| TCAATATATATCACTTTCTCTGGCATCTTCGCTACCGTGTACGTCTCTCTTTCTCTCCCTCCCCTCCTTG | |
| GCTTTTTTCAAGTTCCCACCATAAACGCAGAGGGAGTTAAGAAATGGAGGATAGGAGGTGTGATGTGTTG | |
| TTTCCATGTTCATCATCGGTTGATCCTCGCTTGACAGAGTTTCATGGGGTCGACAACTCTGCTCAGCCGA | |
| CAACATCATCCGAAGAGAAGCCAAGGAGTAAGAAGAAGAAGAAAGAGAGAGAAGCGAGGTACGCGTTCCA | |
| GACAAGAAGCCAGGTTGATATACTGGATGATGGATACAGGTGGAGGAAGTACGGCCAAAAAGCAGTCAAG | |
| AACAATCCATTCCCCAGGAGCTATTATAAGTGCACAGAAGAAGGATGCAGAGTGAAGAAGCAAGTGCAGA | |
| GGCAATGGGGAGACGAAGGAGTGGTGGTGACGACATACCAAGGTGTTCATACACATGCCGTTGATAAACC | |
| CTCTGATAATTTCCACCACATCTTGACACAAATGCACATCTTCCCTCCCTTTTGCTTGAAGGAATGATTA | |
| GAGGAATTGGATTGTAATATTTACTTTCCCAAAAACGTTGGGCTCACACCATCAGACCTTTACTTTTAAA | |
| CTAGCAGCAACTCACATATCTCAAAAATACTAATCCTTATCTTTGTCTTTATGGGACCTTTGAATCCATC | |
| TGCTTTGGTGTCTTAGTCTCGGCTGCCCTGTAATCGAAAGTATATTCATCATCAAATTACCAAACATAAA | |
| GAAGCAATGATGAGTCTATCATCTACAAAAACAATGTTATGTATCCCAAACCTACCGATTATTCCAAAAC | |
| TAGTGACAAGCTAAGGATATTGTGGAGATGAAGATGAGAAAGAGTACGAAAGCTAACTTTGAGGTTTCTT | |
| CTTGGATCCAATTGCGAATATGCTTCACGTTTCGCTTTAGAACGGAGGACGCTTTCTTTGTTAGGCCCAT | |
| TAGCCTGGGCTCTCGTGTTTTTCATAATGTCAAGTCAGCCCAACAAGCCCAAATCTTTACAAAAAGAACC | |
| AAGGACCATGTCATCCGGAATATGGTGATATTATTGGATTATACCATTGGACCATTTAACACAAAAGCAA | |
| ATATGCAACAGAATATATATAGATCACATAACAGTCAGAATCTTCTCAAAAGATATGCTTTCTTCCTTTT | |
| CTATTCTAGAGTGTTTTGATGACATCGGC | |
| SEQ ID NO: 3 AtWRKY45 coding sequence (Start ATG and Stop TGA underlined | |
| and bold) | |
| ATGGAGGATAGGAGGTGTGATGTGTTGTTTCCATGTTCATCATCGGTTGATCCTCGCTTGACAGAGTTTC | |
| ATGGGGTCGACAACTCTGCTCAGCCGACAACATCATCCGAAGAGAAGCCAAGGAGTAAGAAGAAGAAGAA | |
| AGAGAGAGAAGCGAGGTACGCGTTCCAGACAAGAAGCCAGGTTGATATACTGGATGATGGATACAGGTGG | |
| AGGAAGTACGGCCAAAAAGCAGTCAAGAACAATCCATTCCCCAGGAGCTATTATAAGTGCACAGAAGAAG | |
| GATGCAGAGTGAAGAAGCAAGTGCAGAGGCAATGGGGAGACGAAGGAGTGGTGGTGACGACATACCAAGG | |
| TGTTCATACACATGCCGTTGATAAACCCTCTGATAATTTCCACCACATCTTGACACAAATGCACATCTTC | |
| CCTCCCTTTTGCTTGAAGGAATGA | |
| SEQ ID NO: 4 WRKY 45 polypeptide | |
| NP_186846.1 WRKY DNA-binding protein 45 [Arabidopsis thaliana] | |
| medrrcdvlfpcsssvdprltefhgvdnsaqpttsseekprskkkkkerearyafqtrsqvdilddgyrw | |
| rkygqkavknnpfprsyykcteegcrvkkqvqrqwgdegvvvttyqgvhthavdkpsdnfhhiltqmhif | |
| ppfclke | |
| SEQ ID NO: 5 WRKY transcription factor 45 [Camelina sativa] | |
| NCBI Reference Sequence: XP_010482175.1 | |
| 1 | medryqmffp csssvtkvdn stqcgaqpta ssssshqnin tneaekpksk mkkeresrfs |
| 61 | fqtrsqvdil ddgyrwrkyg qkavknnifp rsyykctqeg crvkkqvqrl lgdegvvvtt |
| 121 | yqgvhthpvd kpsdnfhhil tqmhifpsf |
| SEQ ID NO: 6 WRKY transcription factor 75 [Gossypium hirsutum] (Cotton) | |
| 1 | menyqmffpi sapstaaqsl plnmapnsqa fnsfhgnsvd gflglksned liqkpeakdf |
| 61 | mkssqkmekk irkpryafqt rsqvdilddg yrwrkygqka vknnkfprsy yrcthegckv |
| 121 | kkqvqrltkd esvvvttyeg mhthpiqkpt dnfehilsqm qiytpf |
| SEQ ID NO: 7 WRKY transcription factor 45 [Prunus avium] NCBI Reference | |
| Sequence: XP_021823097.1 | |
| 1 | mekyqmffpc ssstssanyd pmipisatnn ittddhhmgm gssqvynyfd grdrssngll |
| 61 | glrssaenhv grevlinkdh hqylqqqysd ltttasanin innvvvgadq npheatnsgn |
| 121 | knkgekktrk pkyafqtrsq vdilddgyrw rkygqkavkn nkfprsyyrc tyqgcnvkkq |
| 181 | vqrltkdegi vvttyegmht hpiekpsdnf ehilnqmqiy tpf |
| SEQ ID NO: 8 WRKY transcription factor 45 [Prunus persica] NCBI | |
| Reference Sequence: XP_007216937.1 | |
| 1 | mekyqmffpc ssstssanyd pmipisatnn ittdghhmgm gssqvynyfd grdqssngll |
| 61 | glrssagnhv grevlinkdh hqylqqqysd ltttasanin innvivgadq npheatnsgn |
| 121 | knkgekktrk pkyafqtrsq vdilddgyrw rkygqkavkn nkfprsyyrc tyqgcnvkkq |
| 181 | vqrltkdegi vvttyegmht hpiekpsdnf ehilnqmqiy tpf |
| SEQ ID NO: 9 WRKY domain class transcription factor [Malus domestica] | |
| GenBank: ADL36856.1 | |
| 1 | msemeasnnm iknnfssqgk sfggsesgea tvrlgmkkgd qkkirkprya fqtrsqvdil |
| 61 | ddgyrwrkyg qkavknnkfp rsyyrcthhg cnvkkqvqrl tkdegvvvtt yegmhshpie |
| 121 | kstdnfehil sqmkiytpf |
| SEQ ID NO: 10 WRKY transcription factor 75 [Rosa chinensis] NCBI | |
| Reference Sequence: XP_024197588.1 | |
| 1 | metyptfyss ssttppaaas slslnmvnsh phhaysndqy qasnnksngf lglmsemevs |
| 61 | nsinsmssis qsmksfgege sntavragmk kgekkirkpr yafqtrsqvd ilddgyrwrk |
| 121 | ygqkavknnk fprsyyrcth qgcnvkkqvq rltkdegvvv ttyegmhshp iekstdnfeh |
| 181 | iltqmqiyts f |
| SEQ ID NO: 11 WRKY transcription factor 45 [Zea mays] NCBI | |
| Reference Sequence: XP_008670731.2 | |
| 1 | menyhmlfgt tathaqpssa agtpssynfi atssasglrr dhdrgqhsgh vhaaggsssp |
| 61 | ssffvaerls hnddsskdgg ggpgpaaags gekeaeaddr paaarrkgek kerrprfafq |
| 121 | trsqvdildd gyrwrkygqk avknnnfprs yyrcthqgcn vkkqvqrlsr degvvvttye |
| 181 | gththpieks ndnfehiltq mqiysgmgst fsrsshdmfh |
| SEQ ID NO: 12 WRKY transcription factor 75 [Glycine max] NCBI Reference | |
| Sequence: XP_003549123.1 | |
| 1 | menysmlfpi snsssypist sgvgssqigy ngqssnaflg lrpsnellgs ddhdnggegg |
| 61 | gdgdgnmlms qisggsntnv sdelggsgns nnkkkgekkv kkpryafqtr sqvdilddgy |
| 121 | rwrkygqkav knnkfprsyy rcthqgcnvk kqvqrltkde gvvvttyegv hthpiekttd |
| 181 | nfehilsqmk iytpf |
| SEQ ID NO: 13 WRKY transcription factor 56 [Oryza sativa Japonica Group] | |
| NCBI Reference Sequence: XP_015615223.1 | |
| 1 | menfpilfat qptssstsss yhfmssssgs hdhrhhhglq aggngggggg slshglfmgs |
| 61 | ssssirmeel snskqagdvv vdggatrsph ggdgdgaagd dggdaqaaaa ggrkkgekke |
| 121 | rrprfafqtr sqvdilddgy rwrkygqkav knnkfprsyy rcthqgcnvk kqvqrlsrde |
| 181 | tvvvttyegt hthpieksnd nfehiltqmh iysgltpssa ahassssplf psaaaaashm |
| 241 | fq |
| SEQ ID NO: 14 WRKY transcription factor 45 [Solanum lycopersicum] NCBI | |
| Reference Sequence: XP_004233585.1 | |
| 1 | mdindvvvgs vsermqndhr nllsvknkki kkprfafqtk sqvdilddgy rwrkygqkav |
| 61 | knnnyprsyy rcthegcnvk kqvqrlskde tvvvttyegm hthpiqkpnd nfeqilhqmh |
| 121 | ifpnppchli n |
| SEQ ID NO: 15 WRKY transcription factor 75-like [Solanum tuberosum] NCBI | |
| Reference Sequence: NP_001275604.1 | |
| 1 | menyatifps assssshhde yislmnskss isddakeell fqgknkagfl glmasmetpr |
| 61 | diitkkdevi ksckkkikkp ryafqtrsqv dilddgyrwr kygqkavknn kfprsyyrct |
| 121 | hqgcnvkkqv qrlskdeevv vttyegmhsh pidkstdnfe hilsqmqiyt sf |
| SEQ ID NO: 16 WRKY DNA-binding protein 75 [Arabidopsis thaliana] NCBI | |
| Reference Sequence: NP_196812.1 | |
| 1 | megydngsly apflslkshs kpelhqgeee sskvrsegcs ksvesskkkg kkqryafqtr |
| 61 | sqvdilddgy rwrkygqkav knnkfprsyy rctyggcnvk kqvqrltvdq evvvttyegv |
| 121 | hshpiekste nfehiltqmq iyssf |

SEQ ID NO: 17 AtWRKY45_1 forward primer
ATGGAGGATAGGAGGTGTGAT
SEQ ID NO: 18 AtWRKY45_1 reverse primer
TCATTCCTTCAAGCAAAAGGGA
SEQ ID NO: 19 35S promoter specific forward primer
GGAGAGGACCTCGACTCTAGA

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Analysis of Arabidopsis thaliana WRKY45 (AtWRKY45)

Plants are continuously challenged by abiotic (e.g. drought and salt) and biotic (e.g. insects) stresses. WRKY family of transcription factors (TFs) are one of the largest families of regulatory proteins in plants that have been shown to play important roles in responses to biotic and abiotic stresses (Wang et al. 2014; Rinerson et al. 2015; Chen et al. 2017; Guo et al., 2018). Besides higher plants (rice, soybean, Arabidopsis, tobacco), WRKY TFs have also been identified in protists like Giardia lamblia and Dictyostelium discoideum, unicellular green algae like Chlamydomonas reinhardtii, multicellular green algae like Klebsormidium flaccidum and mosses (Physcomitrella patens) (Robatzek and Somssich, 2001). Presence of WRKY genes in lower life forms indicates an ancient origin of this gene family.

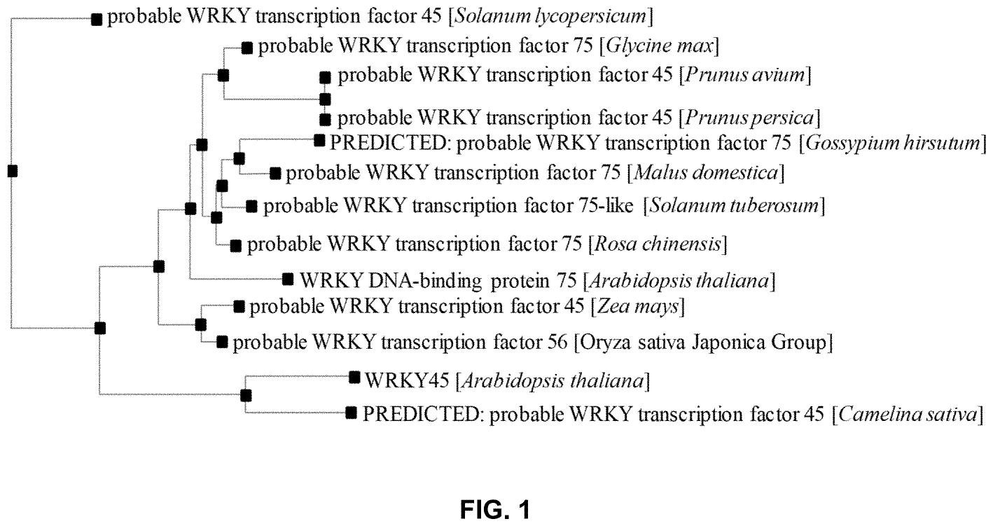

Arabidopsis thaliana, a relative of plants in the Crucifer family that includes cauliflower, cabbage, turnip, mustard, and canola contains over 70 WRKY genes. The WRKY45 (At3g01970) gene in Arabidopsis thaliana (AtWRKY45) was shown to be involved in phosphate starvation and age-triggered senescence (Wang et al. 2014; Chen et al. 2017). The present example provides experimental evidence, through analysis of AtWRKY45 expression, that AtWRKY45 is involved in controlling infestation by the green peach aphid (Myzus persicae), which is recognized as the 3rd most damaging insect pest of plants, including agricultural and horticultural plants in over 50 plant families (Klein Koch and Waterhouse, 2000). FIG. 1 shows a phylogeny tree illustrating similarity between Arabadopsis WRK45 and WRKY45 in other plants. BLAST identity results are shown in Table 1, below.

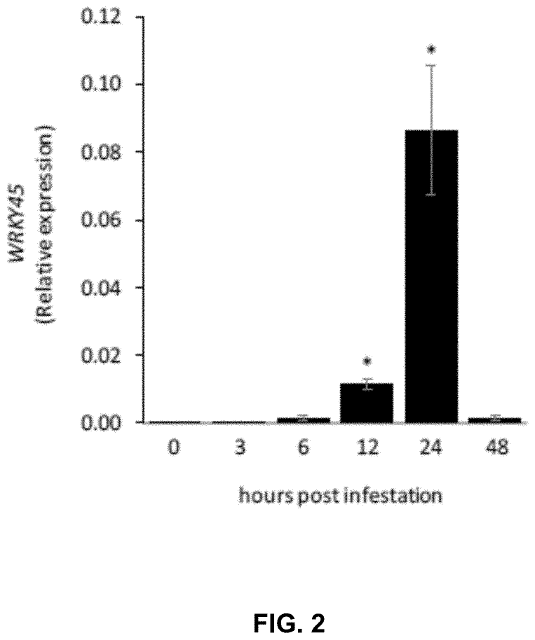

As illustrated by the graph in FIG. 2, AtWRKY45 expression in Arabidopsis is transiently upregulated in response to green peach aphid (GPA infestation). FIG. 2 shows a time course analysis of AtWRKY45 expression in Arabidopsis in response to GPA infestation. AtWRKY45 expression was analyzed at 0, 3, 6, 12, 24 and 48 h post infestation with GPA (n=3). Error bars indicate standard error. Asterisks above bars indicate values that are significantly different from the 0 h (P<0.05; ttest). Expression of AtWRKY45s relative to the expression of At1g07940, which was used as a control for the real time RT-PCR.

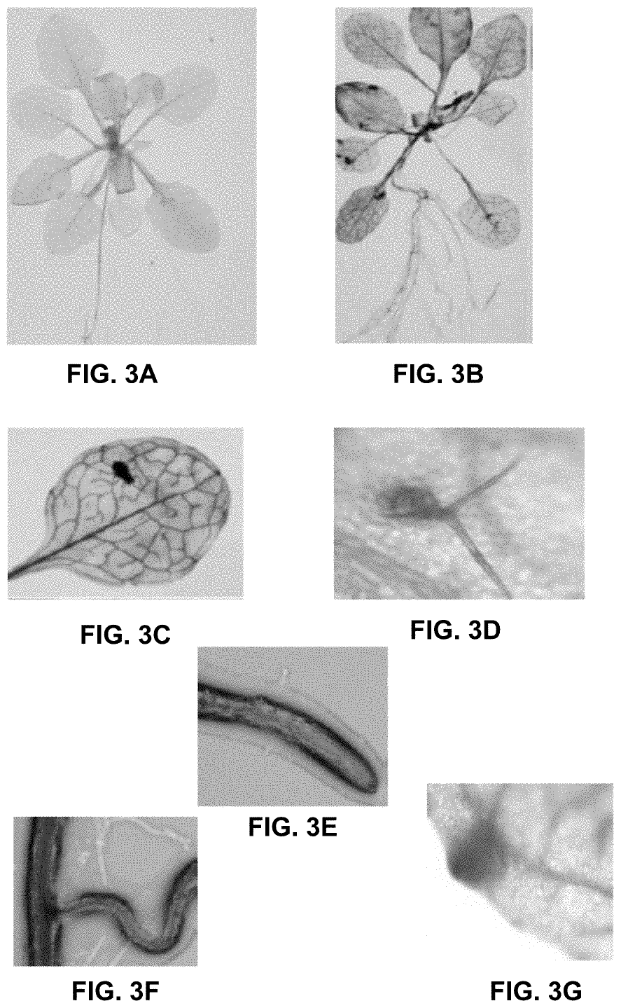

Analysis also demonstrated that AtWRKY45 promoter activity was stimulated in leaves and roots of GPA infested plants. As illustrated in FIGS. 3A-3G, the GUS reporter allows visual staining to study where the construct is expressed. In this case GUS expression was driven from the AtWRKY45 promoter thus allowing us to see where the AtWRKY45 promoter activity (and hence by extrapolation, AtWRKY45 expression) is active. FIGS. 3B-3G illustrate that AtWRKY45 promoter activity is high in leaves and roots of GPA-infested plants. Twenty six days old AtWRKY45pro:uidA transgenic plants in which the AtWRKY45 promoter was used to drive expression of the GUS reporter encoded by the UidA gene derived from Escherichia coli were analyzed for GUS activity before and 24 hour after GPA infestation. FIG. 3A illustrates a un-infested (Control) AtWRKY45pro:uidA plant; FIG. 3B shows GPA infested AtWRKY45pro:uidA plant; FIG. 3C illustrates strong expression of AtWRKY45 in leaf vasculature; FIG. 3D shows WRKY45 expression in a trichome; FIG. 3E depicts AtWRKY45 expression in the columella root cap cells at the tip of the root; FIG. 3F shows AtWRKY45 expression in root tissue, with strong expression in vasculature; and FIG. 3G illustrates AtWRKY45 expression in hydathodes. This expression analysis indicating that AtWRKY45 expression is upregulated in roots in response to stress suggests that its function is likely exerted in roots, resulting in a protective effect across the plant.

TABLE 1
AtWRKY45 BLAST identity results

| | | GenBank | | % query | sequence | | SEQ |
|---|---|---|---|---|---|---|---|
| Query | Hit | ACCESSION | % identity | covered | length | evalue | ID NO. |
| Arabidopsis | WRKY DNA-binding | NP_186846.1 | 100 | 100 | 147 | ######## | 4 |
| WRKY45 | protein 45 | | | | | | |
| | [Arabidopsis thaliana] | | | | | | |
| Arabidopsis | WRKY transcription | XP_010482175.1 | 72.785 | 97.27891156 | 149 | 4.17E−71 | 5 |
| WRKY45 | factor 45 [Camelina | | | | | | |
| | sativa] | | | | | | |
| Arabidopsis | WRKY transcription | XP_016725404.1 | 64.615 | 87.75510204 | 166 | 5.71E−54 | 6 |
| WRKY45 | factor 75 [Gossypium | | | | | | |
| | hirsutum] | | | | | | |
| Arabidopsis | WRKY transcription | XP_021823097.1 | 68.067 | 80.27210884 | 223 | 6.41E−54 | 7 |
| WRKY45 | factor 45 [Prunus | | | | | | |
| | avium] | | | | | | |
| Arabidopsis | WRKY transcription | XP_007216937.1 | 68.067 | 80.27210884 | 223 | 6.99E−54 | 8 |
| WRKY45 | factor 45 [Prunus | | | | | | |
| | persica] | | | | | | |
| Arabidopsis | WRKY domain class | ADL36856.1 | 74.747 | 66.66666667 | 190 | 1.81E−51 | 9 |
| WRKY45 | transcription factor | | | | | | |
| | [Malus domestica] | | | | | | |
| Arabidopsis | WRKY transcription | XP_024197588.1 | 74.257 | 68.02721088 | 191 | 2.27E−51 | 10 |
| WRKY45 | factor 75 [Rosa | | | | | | |
| | chinensis] | | | | | | |
| Arabidopsis | WRKY transcription | XP_008670731.2 | 75.248 | 68.02721088 | 220 | 5.73E−51 | 11 |
| WRKY45 | factor 45 [Zea mays] | | | | | | |
| Arabidopsis | WRKY transcription | XP_003549123.1 | 79.348 | 61.9047619 | 195 | 6.34E−51 | 12 |
| WRKY45 | factor 75 [Glycine | | | | | | |
| | max] | | | | | | |
| Arabidopsis | WRKY transcription | XP_015615223.1 | 76 | 67.34693878 | 242 | 1.90E−50 | 13 |
| WRKY45 | factor 56 [Oryza | | | | | | |
| | sativa Japonica | | | | | | |
| | Group] | | | | | | |
| Arabidopsis | WRKY transcription | XP_004233585.1 | 73.077 | 69.3877551 | 131 | 1.15E−49 | 14 |
| WRKY45 | factor 45 [Solanum | | | | | | |
| | lycopersicum] | | | | | | |
| Arabidopsis | WRKY transcription | NP_001275604.1 | 76.087 | 61.9047619 | 172 | 2.56E−48 | 15 |
| WRKY45 | factor 75-like | | | | | | |
| | [Solanum tuberosum] | | | | | | |
| Arabidopsis | WRKY DNA-binding | NP_196812.1 | 75 | 61.9047619 | 145 | 2.09E−46 | 16 |
| WRKY45 | protein 75 | | | | | | |
| | [Arabidopsis thaliana] | | | | | | |

### Example 2

### Overexpression of Recombinant Arabidopsis thaliana WRKY45 in Arabidopsis and Response to Abiotic and Biotic Stressors

The present example demonstrates that WRKY45 expression, when increased, results in curtailment of aphid infestation and confers enhanced tolerance to drought and salt stress. WRKY45 overexpression also promotes recovery from drought when plants are re-watered. Drought and salinity are two major abiotic stressors of plants that impact agricultural economics. For example, the 1998 and 2012 drought in the US caused agricultural losses to the extent of $40 billion (Rippey 2015). Similarly, ˜20% of the world's agricultural land is affected by salt stress each year which leads to enormous amount of yield loss (Guo et al. 2018).

Preparation of AtWRKY45 Arabidopsis Plants

The WRKY45 coding sequence (CDS) was amplified from a clone (U84666) available from Arabidopsis Biological Resource Center (https://abrc.osu.edu/) using the FP_WRKY45_1 (SEQ ID NO: 17) and RP_WRKY45_1 (SEQ ID NO: 18) primer pair. The CDS was then cloned into the pCR_8/GW/TOPO vector (Life Technologies; www.lifetechnologies.com), from which the AtWRKY45 CDS was mobilized to the destination vector pMDC32 (Curtis and Grossniklaus., 2003) with the help of LR clonase recombination reaction (Life Technologies; www.lifetechnologies.com). The resultant plasmid pMDC32:WRKY45 contains the AtWRKY45 CDS between the Cauliflower mosaic virus 35S gene promoter and the Agrobacterium tumefaciens nos transcription terminator, such that in planta the AtWRKY45 CDS is expressed from the ubiquitously expressed 35S promoter. The pMDC32:WRKY45 construct was then transformed into Agrobacterium tumefaciens strain GV3101. This Agrobacterium strain was used to transform the wild-type Arabidopsis accession Col plants using the floral-dip method (Zhang et al., 2006). The transformants were selected on ½ strength Murashige and Skoog agar plates supplemented with hygromycin (25 mg/L). Presence of the transgene in the selected transformants was confirmed by genotyping using the 35S promoter specific forward primer 5′-GGAGAGGACCTCGACTCTAGA-3′ (SEQ ID NO: 19) and the WRKY45 specific reverse primer 5′-TCATTCCTTCAAGCAAAAGGGA-3′ (SEQ ID NO: 18). PCR conditions used to amplify the product was denaturation at 95° C. for 5 min, followed by 30 cycles of 95° C. for 30 s, 58° C. for 30 s and 72° C. for 45 s, with a final extension of 72° C. for 5 min. T3 generation plants were used for all the experiments.

Overexpression of AtWRKY45 in Arabidopsis was Shown to Limit GPA Reproduction

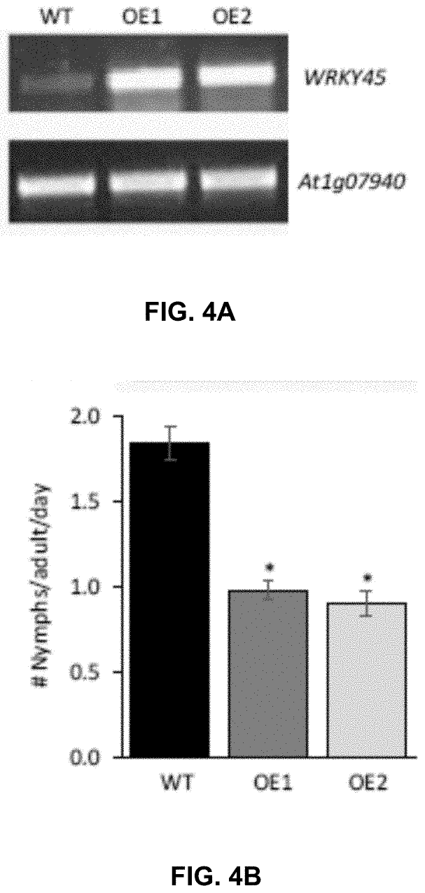

Overexpression of WRKY45 coding sequence from the CaMV 35S promoter in transgenic Arabidopsis thaliana plants results in reduced infestation that is associated with reduced fecundity (reproduction) of the green peach aphid (GPA). FIG. 4A illustrates expression of AtWRKY45 in OE1 and OE2, two independently derived 35S:WRKY45 transgenic lines, as well as in wild type Arabidopsis. At1g07940 is a control gene that encodes an elongation factor, which is used to monitor quality of RT-PCR reaction for each sample (thus, expression of At1g07940 should be comparable in all samples as shown in FIG. 4A). Compared to the wild-type (WT), AtWRKY45 expression is higher in the OE1 and OE2 lines (FIG. 4A) and the number of progeny produced by each green peach aphid per day is lower in the OE1 and OE2 liens compared to the WT plant (FIG. 4B).

AtWRKY45 Overexpression in Arabidopsis Promotes Drought Tolerance and Recovery

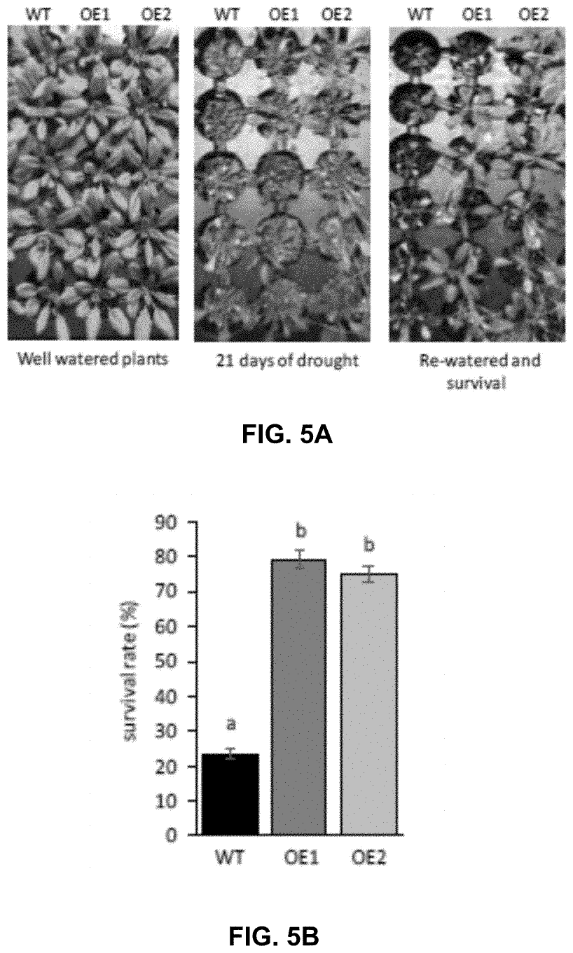

Two-week old WRKY45 overexpressing lines OE1 and OE2 and the non-transgenic wild-type Arabidopsis plants were exposed to drought stress for three weeks by withholding water. At the end of three weeks of drought, plants were watered, and the recovery monitored. Plants were photographed before drought initiation (FIG. 5A, left panel), at the end of 3 week of water withholding (FIG. 5A, center panel) and after recovery associated with rewatering (FIG. 5A, right panel).

Survival rate (%) in WT Col and WRKY45-OE lines after recovery from drought is shown in FIG. 5B. Error bars indicate SE. ANOVA following the General Linear Model followed by Tukey's multiple comparison test was used to determine statistical significance of difference in survival between the three lines. Different letters above bars indicate values that are significantly different from each other. Both figures demonstrate that while a majority of the WRKY45 overexpressing plants survived the drought and recovered, a large proportion of non-transgenic WT plants did not recover.

AtWRKY45 Overexpression in Arabidopsis Enhances Stomatal Closure During Drought

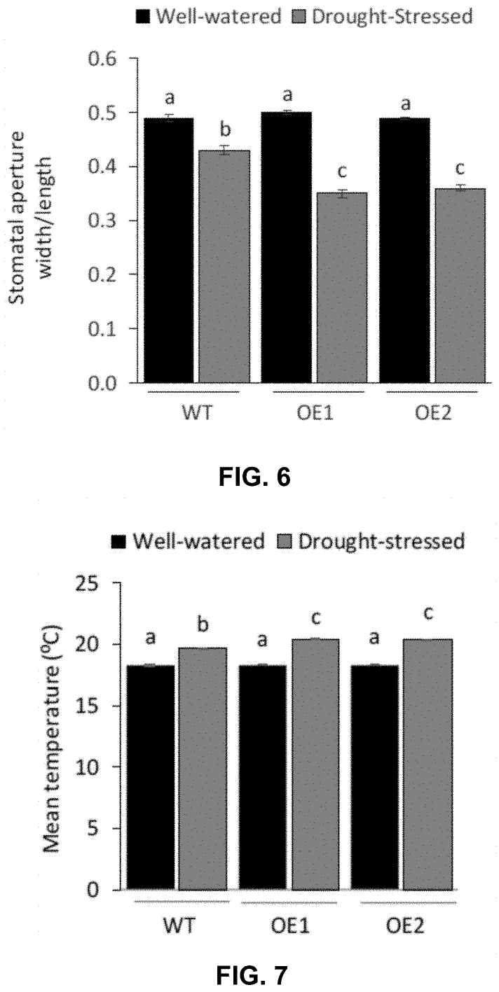

AtWRKY45-OE and WT Col plants were exposed to drought stress by withholding water for two weeks and well-watered plants were used as a control. Leaves were observed under the microscope at 40×. A stomatal aperture ratio of plants exposed to 2 weeks of drought (n=10) was calculated. FIG. 6 shows that stomatal aperture ratio of well-watered plants was similar in WT and AtWRKY45-OE plants; however, stomatal closure was greater during drought stress in the AtWRKY45-OE plants. General Linear Model followed by Tukey's multiple comparison test was used to determine significance of mean values (P≤0.05). Different letters above bars denote values that are significantly different from each other.

AtWRKY45 overexpressing lines also demonstrated reduced transpiration under drought stress. Leaf thermal imaging, used to monitor leaf temperature as an indirect measure of leaf transpiration, was carried out on AtWRKY45 overexpressing Arabidopsis transgenic lines OE1 and OE2 compared to the non-transgenic wild-type Arabidopsis accession Columbia plants. Plants were exposed to drought conditions and compared, as control, to corresponding well-watered plants. A thermal camera was used to measure temperature (degree Celsius) of leaves from different genotypes as illustrated in FIG. 7. Error bars indicate standard error. ANOVA following the General Linear Model followed by Tukey's multiple comparison test was used to determine significance of mean values (P≤0.05); different letters above the bar indicate the values are significantly different. Under drought conditions there is generally an increase in temperature of plants due to reduced transpiration resulting from closure of stomates. In case of AtWRKY45 overexpressing lines the temperature increases are higher, indicating more robust closure of stomates thus minimizing water loss.

RNA seq analysis conducted demonstrated that WRKY45 overexpression results in upregulation of a variety of genes that are associated with water-related stress, thus suggesting that WRKY45 is a master regulator of processes involved in controlling water utilization in plants. Stomatal aperture size measurements, combined with thermal imaging, indicate that WRKY45 overexpressing plants are better able to control stomatal aperture size and hence water loss when stressed.

AtWRKY45 Overexpression in Arabidopsis Promotes Salt Tolerance

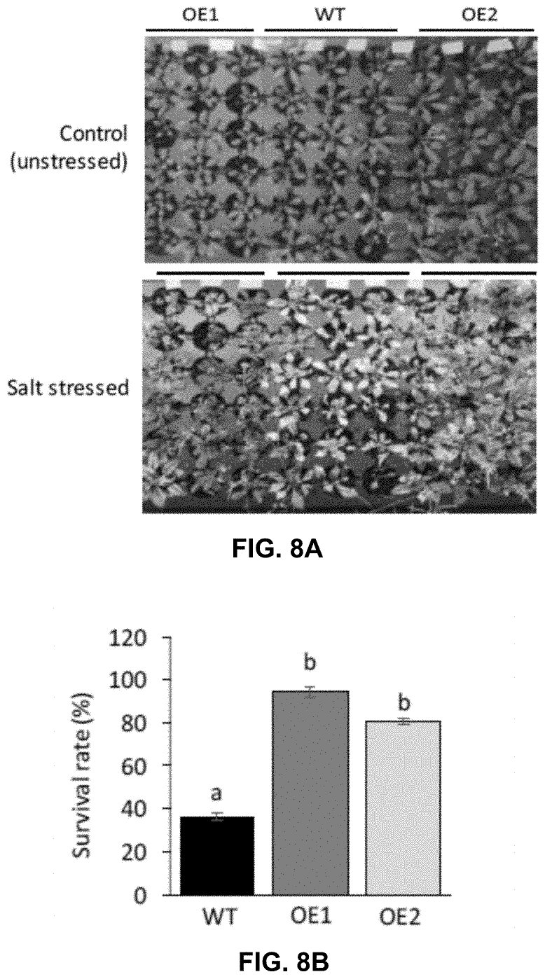

AtWRKY45 overexpressing (OE) lines OE1 and OE2 and non-transgenic wild-type (WT) Arabidopsis were exposed to salt stress (NaCl; 250 mM) for 3 weeks and compared to control (unstressed) plants that received water. FIG. 8A illustrates the phenotype of the AtWRKY45 OE lines vs the WT Arabidopsis plants after a 3-week exposure to salt (bottom panel) vs control (top panel). While the WT plants have become highly chlorotic and have died, the WRKY45 overexpressing lines show increased survival and retain chlorophyll (green coloration).

FIG. 8B is a graph of the survival rate of AtWRKY45 OE lines OE1 and OE2 and non-transgenic WT Arabidopsis plants after the 3-week exposure to salt (NaCl; 250 mM). The graph illustrates significantly improved survival rate in the OE lines. Error bars indicate standard error. ANOVA following the General Linear Model followed by Tukey's multiple comparison test was used to determine significance of mean values (P≤0.05). Different letters above the bars indicate values that are significantly different from each other.

AtWRKY45 Overexpression in Arabidopsis Results in Constitutively Higher Expression of Genes Associated with Stress Response

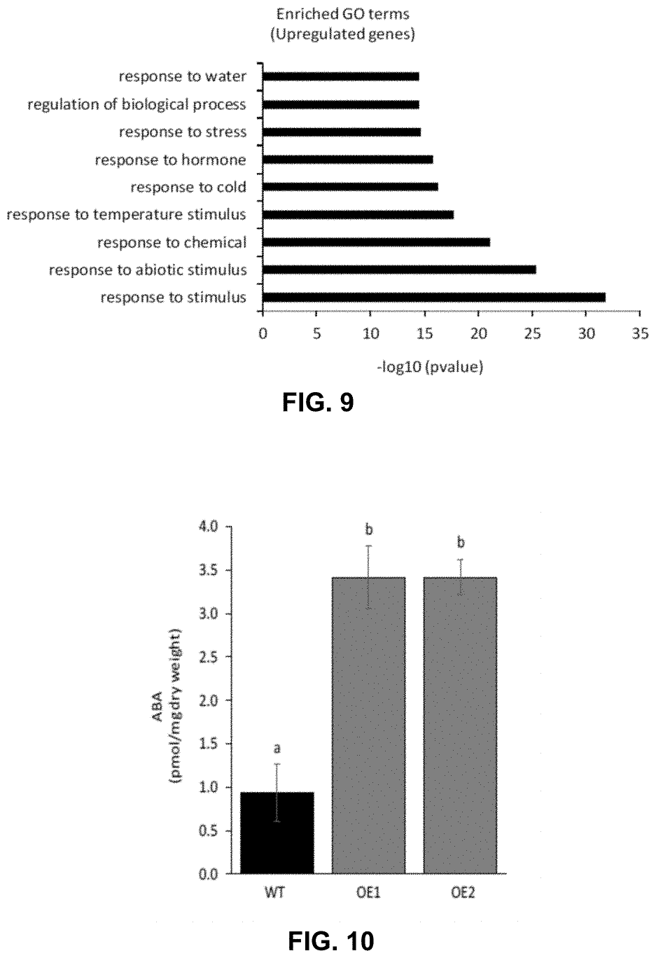

Gene ontology of genes that are expressed at higher levels in WRKY45 overexpressing Arabidopsis transgenic plants compared to the wild-type (WT) control plant is illustrated in FIG. 9. The Gene Ontology (GO) study was conducted on genes that are differentially expressed in AtWRKY45 overexpressing plants using Agrigo (http://systemsbiology.cau.edu.cn/agriGOv2/). GO classifies genes based on their functions and properties (http://geneontology. org/). The top ten over-represented GO terms among the genes upregulated in WRKY45-OE (p-value<0.05) were those associated with response to different stimuli like, abiotic stress (temperature, cold, chemical, water), hormones, cell regulation and cell communication.

Experiments also show that AtWRKY45 overexpression in Arabidopsis results in constitutively elevated levels of abscisic acid (ABA), a plant hormone involved in promoting adaptation to stress. FIG. 10 illustrates that levels of the stress associated hormone ABA is higher in AtWRKY45 overexpressing Arabidopsis lines OE1 and OE2 than in WT plants. This provides some insight on possible underlying mechanisms of how AtWRKY45 expression might be affecting drought and salt tolerance.

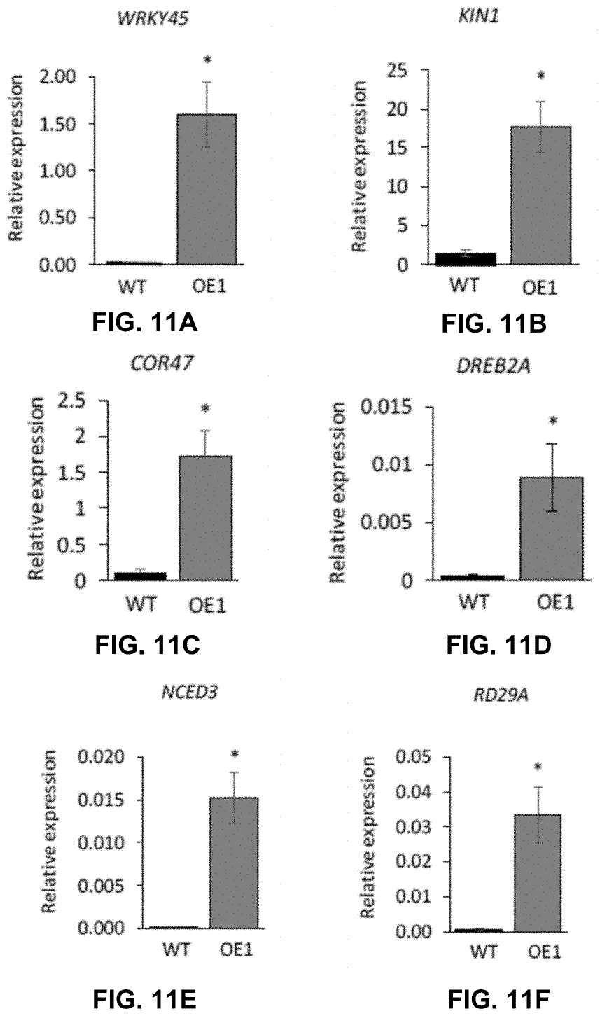

Expression of abiotic stress associated genes is constitutively higher in AtWRKY45 overexpressing Arabidopsis plants compared to the WT. qRT-PCR confirmation of abiotic stress associated genes in the AtWRKY45 OE1 compared to the wild-type plant. RNA extracted from leaves of WT accession Columbia and AtWRKY45 overexpressing plants were used to validate the RNA-seq results. FIGS. 11A-11F illustrate relative expression in WT and AtWRKY45OE1 lines of AtWRKY45 (FIG. 11A) and 5 stress associated genes: KIN1 (FIG. 11B), COR47 (FIG. 11C), DREB2A (FIG. 11D), NCED3 (FIG. 11E), and RD29A (FIG. 11F). Expression of all genes is relative to the expression of At1g07940 that encodes a Tu family elongation factor. Asterisks denote values that are significantly different (p<0.05; t-test) from the WT. Error bars represent standard error (n=4).

This data and results above indicate that Arabidopsis thaliana WRKY45 gene overexpression results in overexpression of various stress related genes as well as hormones involved in stress adaptation. Results also demonstrate that recombinant versions of the gene can be utilized, through overexpression in a plant, to enhance tolerance to multiple stress in plants, including resistance to an important agricultural pest, as well as drought and salinity.

### Example 3

### Expression of Recombinant Arabidopsis thaliana WRKY45 in Tomato Plants and Response to Abiotic and Biotic Stressors

In the present example, tomato plants (Solanum lycopersicum) variety Moneymaker were transformed with the Arabidopsis thaliana WRKY45 CDS (AtWRKY45) expressed from the Cauliflower mosaic virus 35S gene promoter. Agrobacterium tumefaciens was used for transforming tomato. Several independent transgenic lines were identified. These lines and a non-transformed S. lycopersicum Moneymaker control were tested for resistance/tolerance of two abiotic (drought and salinity) stressors and one biotic (green peach aphid) stressor. Overexpression of AtWRKY45 was found to confer increased tolerance to all three stressors over the wild-type plant.

### Materials & Methods

Creating Transgenic Tomato Plants Expressing AtWRKY45

Tomato transformation used the same construct that was used to transform Arabidopsis (see Example 2). The AtWRKY45 CDS is driven from the Cauliflower mosaic virus 35S gene promoter with terminator sequence derived from the Agrobacterium tumefaciens nos gene. Hygromycin was used as the selective marker to select positive transformants.

Transformation of tomato plants was achieved through tissue culture, and several independent transformants were picked up on the selective media. Since these transformants are derived from different calli, they are independent transformation events of a separate cell being transformed. In each of these independent transformation events stochastically the recombinant construct could have inserted at a separate position in the genome. Seeds collected from each of these transformed plants were maintained as separate lines (not mixed) so that their effects on stress phenotypes could be monitored independently. The bioassays were performed on plants derived from each of these independent lines, and data for each transgenic line was recorded separately to account for any differences in expression of the transgene in these independent lines, or issues related to where the insertion occurred. Collecting data from independent lines ensures that the observed effect on the phenotype of interest (e.g., drought and salt tolerance and resistance to aphid) is indeed an effect of the transgene and not due to other factors.

These WRKY45 expressing transgenic tomato plants (progeny from one or more independent lines #5, 7, 8, 22 and 27) were tested for their response to drought, salinity and infestation by the green peach aphid (Myzus persicae).

Aphid Resistance Assay

Five adult insects were released on tomato cotyledons, and the number of nymphs produced per adult insect was calculated at 72 h post release of the insect on the tomato plants. Three independently derived AtWRKY45 lines (Line #5, line #8, and line #22) were tested along with a non-transgenic control.

Drought Tolerance/Recovery Assay

Plants were exposed to drought conditions for 12 days, after which they were rewatered. Photos were taken 17 days after rewatering, and growth measurements were also taken at this time. Three independently derived AtWRKY45 lines (Line #5, line #7, and line #8) were tested along with a non-transgenic control.

Salt Stress Assay

Two independent AtWRKY45 tomato lines (line #8 and line #27 were evaluated along with a non-transgenic control. Plants were treated with 200 mM salt (sodium chloride) for 36 days. Images (control and line 27) were taken 19 days after initiation of salt stress. Stem diameter of all three lines was measured at 36 days as an indicator of growth and tolerance.

Results & Discussion

Aphid Resistance

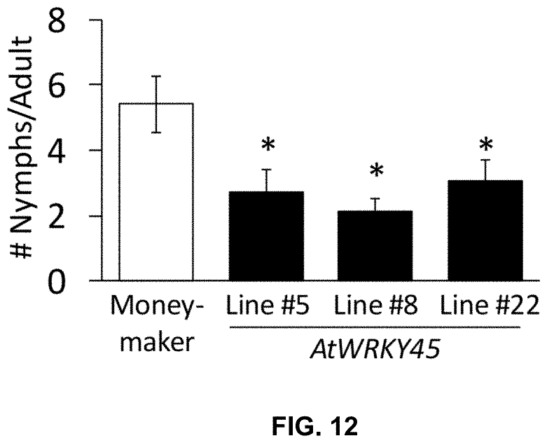

All three of the AtWRKY45 expressing transgenic tomato plants (AtWRKY45 expressing tomato) exhibited statistically significant enhanced resistance to the green peach aphid. Insect fecundity (number of progeny produced by each aphid over a period of time) was lower on the WRKY45 expressing transgenic tomato than on the non-transgenic tomato plants. FIG. 12 illustrates that 40-50% reduction in insect fecundity was observed in the AtWRKY45 expressing tomato plants. Asterisks indicate values that are significantly different (P<0.05) than the non-transgenic Moneymaker plants. Thus, it was demonstrated that AtWRKY45 CDS expression in tomato plants results in higher level of resistance to the green peach aphid (Myzus persicae) than non-transgenic tomato cultivar Moneymaker plants.

Drought Tolerance

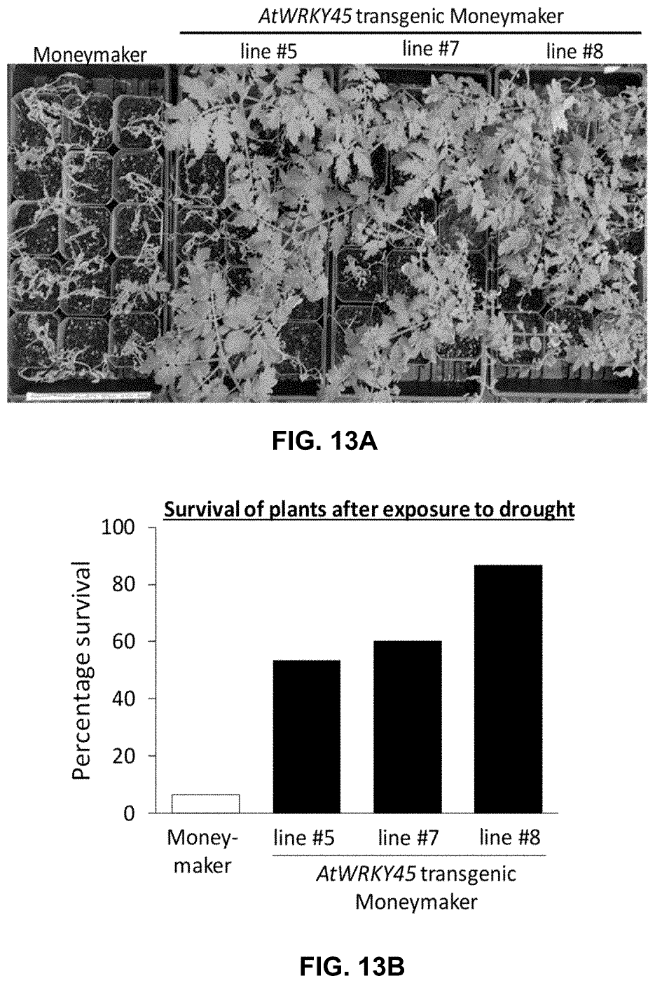

The AtWRKY45 expressing transgenic tomato also exhibited enhanced tolerance to drought. Recovery from drought was more robust in the AtWRKY45 expressing transgenic tomato plants than the non-transgenic variety Moneymaker plants once watering was re-initiated after drought. As illustrated in FIGS. 13A and 13B, while the non-transgenic Moneymaker plants show poor recovery (<10% survival), the transgenic AtWRKY45 expressing tomato line #5, 7 and 8 exhibit robust recovery (50-90% recovery). The photo in FIG. 13A shows that nearly all control Moneymaker plants died even after watering was resumed. This demonstrates that AtWRKY45 conferred robust drought resistance and recovery compared to the non-transgenic control Moneymaker plants.

Salt Stress

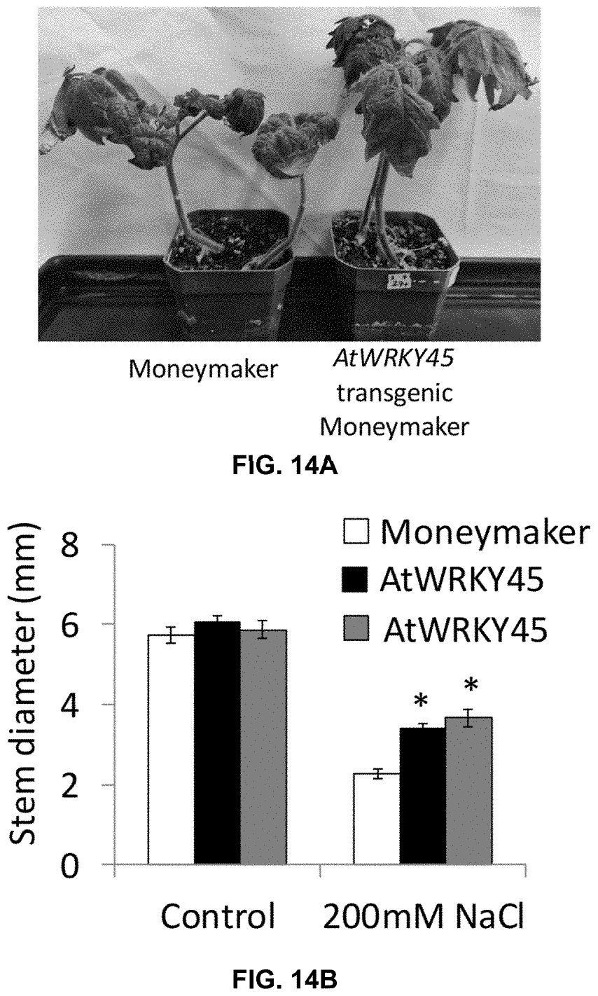

Stem diameter, used as an indicator of growth and vigor, was significantly larger in the salt stressed AtWRKY45 expressing transgenic tomato plants (lines 8 and 27) compared to the non-transgenic Moneymaker plants (FIGS. 14A-4B). Asterisks indicate values that are significantly different from the salt stressed Moneymaker. The photograph in FIG. 14A shows severe curing of leaves in salt stressed non-transgenic plant as compared to one of the salt-stressed AtWRKY45 expressing transgenic plants (Line 27). FIG. 14B illustrates that while there is no significant difference in growth (stem diameter) between non-transgenic and AtWRKY45 expressing transgenic tomato plants under control (no salt) conditions, the AtWRKY45 expressing transgenic plants showed significantly larger stem diameter when exposed to 200 nM sodium chloride for 36 days when compared to similarly salt stressed non-transgenic tomato plants. Thus, compared to the non-transgenic tomato Moneymaker plants, the AtWRKY45 expressing transgenic tomato withstood salinity stress better.

These results confirm that like in Arabidopsis, constitutive expression of the Arabidopsis WRKY45 coding sequence confers more effective control of aphid infestation and in addition confers improved tolerance to drought and salinity stress in tomato plants.

### REFERENCES

Chen, L., Xiang, S., Chen, Y., Li, D., and Yu, D. (2017) Arabidopsis WRKY45 interacts with the DELLA protein RGL1 to positively regulate age-triggered leaf senescence. Mol. Plant 10. 1174-1189.
Guo R, Qiao H, Zhao J Wang X, Tu M, Guo C, Wan R, Li Z, Wang X (2018)The Grape VIWRKY3 Gene Promotes Abiotic and Biotic Stress Tolerance in Transgenic Arabidopsis thaliana. Frontiers in Plant Science. Doi: 10.3389/fpls.2018.00545
Klein Koch, C. and Waterhouse, D. F. (2000). The distribution and importance of arthropods associated with agriculture and forestry in Chile. Monographs. https://www.aciar.gov.au/node/7506
Rippey, B. R. (2015) The U.S. drought of 2012. Weather and Climate Extremes 10, 57-64.
Rinerson C I, Rabara R C, Tripathi P, Shen Q J, Rushton R J (2015) The evolution of WRKY transcription factors. BMC Plant Biol. 15: 66.
Robatzek S, Somssich I E (2001) A new member of the Arabidopsis WRKY transcription factor family, AtWRKY6, is associated with both senescence- and defense-related processes. Plant J 28:123-133.
Wang, H., Xu, Q., Kong, Y. H., Chen, Y., Duan, J. Y., Wu, W. H., and Chen, Y. F. (2014) Arabidopsis WRKY45 transcription factor activates PHOSPHATE TRANSPORTER 1;1 expression in response to phosphate starvation. Plant Physiol. 164, 2020-2029.
Curtis M D, Grossniklaus U (2003) A gateway cloning vector set for high-throughput functional analysis of genes in planta. Plant Physiol 133: 462-469.
Zhang X, Henriques R, Lin S S, Niu Q W, Chua N H (2006) Agrobacterium-mediated transformation of Arabidopsis thaliana using the floral dip method. Nat Protoc 1: 641-646.

We claim:

1. A recombinant polynucleotide that encodes a WRKY polypeptide, the recombinant polynucleotide comprising:
a WRKY45 polynucleotide having a sequence that is about 50-100% identical to any one of SEQ ID NOs: 1-3, and
at least one heterologous polynucleotide sequence operatively linked to the WRKY45 polynucleotide.

2. The recombinant polynucleotide of claim 1, wherein the at least one heterologous polynucleotide sequence comprises a regulatory polynucleotide sequence, a selectable marker polynucleotide, or both.

3. A vector comprising the recombinant polynucleotide of claim 1.

4. The vector of claim 4, wherein the at least one heterologous polynucleotide sequence comprises a regulatory polynucleotide sequence, a selectable marker polynucleotide, or both.

5. A cell comprising a recombinant polynucleotide of claim 1 or a vector comprising the recombinant polynucleotide of claim 1.

6. The cell of claim 5, wherein the cell is a plant, bacteria, yeast, or fungus cell. The cell of claim 5, wherein the cell is a plant cell.

8. The cell of claim 7, wherein the cell is selected from the group of plant cells consisting of: Arabidopsis, rice, wheat, barley, cotton, rose, china rose, apple, camelina, peach, maize, tobacco, soybean, Brassicas, tomato, potato, bell pepper, alfalfa, chickpea, sugarcane, sorghum, eggplant, sweet pepper, papaya, tobacco, cannabis, and canola.

9. A transgenic plant comprising:
a plurality of cells, wherein one or more of the plurality of cells comprises a recombinant polynucleotide of claim 1 or a vector comprising the recombinant polynucleotide of claim 1.

10. The transgenic plant of claim 9, wherein transgenic plant expresses an increased amount of a WRKY transcription factor protein as compared to a non-transgenic control.

11. The transgenic plant of claim 9, wherein the transgenic plant is selected from the group of plants consisting of: Arabidopsis, rice, wheat, barley, cotton, rose, china rose, apple, camelina, peach, maize, tobacco, soybean, Brassicas, tomato, potato, bell pepper, alfalfa, chickpea, sugarcane, sorghum, eggplant, sweet pepper, papaya, tobacco, cannabis, and canola.

12. The transgenic plant of claim 9, wherein the transgenic plant has increased tolerance to an abiotic stressor.

13. The transgenic plant of claim 12, wherein the abiotic stressor is salinity or drought.

14. The transgenic plant of claim 9, wherein the transgenic plant has increased tolerance to a biotic stressor.

15. The transgenic plant of claim 14, wherein the biotic stressor is an insect.

16. The transgenic plant of claim 15, wherein the insect is a green peach aphid Myzus persicae.

17. A method of increasing tolerance to an abiotic or biotic stressor in a plant, the method comprising:
integrating into the genome of at least one cell of a plant a recombinant polynucleotide of claim 1 or a vector comprising the recombinant polynucleotide of claim 1 such that the recombinant polynucleotide is expressed in the plant cell; and
growing said plant, wherein the recombinant polynucleotide is overexpressed in the plant relative to a wild-type plant and wherein the plant has increased tolerance, as compared to a non-transgenic control plant or wild type plant, to one or more abiotic stressors, one or more biotic stressors, or a combination thereof.

18. The method of claim 17, wherein the plant is selected from the group of plants consisting of: Arabidopsis, rice, wheat, barley, cotton, rose, china rose, apple, camelina, peach, maize, tobacco, soybean, Brassicas, tomato, potato, bell pepper, alfalfa, chickpea, sugarcane, sorghum, eggplant, sweet pepper, papaya, tobacco, cannabis, and canola.

19. The method of claim 17, wherein the abiotic stressor is salinity, drought, or both and wherein the abiotic stressor is an insect.

20. A recombinant polynucleotide that encodes a WRKY polypeptide, the recombinant polynucleotide comprising:
a WRKY45 polynucleotide having a sequence that is about 50-100% identical to any one of SEQ ID NOs: 2-3.


SEQUENCE LISTING (19 sequences)

SEQ ID NO: 1 - DNA, 1461 bp, Arabidopsis thaliana
gtttgaaatttgaatccattgaaccaaaatttgaaggagttgcatatataataatataaatcagaatgatgtagccgccacacctttttgtttccacaaaactctttttctgtgatggatccgctaatgtagccatattttcaatatatatcactttctctggcatcttcgctaccgtgtacgtctctctttctctccctcccctccttggcttttttcaagttcccaccataaacgcagagggagttaagaaatggaggataggaggtgtgatgtgttgtttccatgttcatcatcggttgatcctcgcttgacagagtttcatggggtcgacaactctgctcagccgacaacatcatccgaagagaagccaaggagtaagaagaagaagaaagagagagaagcgaggtacgcgttccagacaagaagccaggttgatatactggatgatggatacaggtggaggaagtacggccaaaaagcagtcaagaacaatccattccccaggtacgtacttaatcatcaatgaatgatactgtatgcttgctatggtgatcaaataagagactcatatacatacatgtaaaatatgaatatatatatatataggagctattataagtgcacagaagaaggatgcagagtgaagaagcaagtgcagaggcaatggggagacgaaggagtggtggtgacgacataccaaggtgttcatacacatgccgttgataaaccctctgataatttccaccacatcttgacacaaatgcacatcttccctcccttttgcttgaaggaatgattagaggaattggattgtaatatttactttcccaaaaacgttgggctcacaccatcagacctttacttttaaactagcagcaactcacatatctcaaaaatactaatccttatctttgtctttatgggacctttgaatccatctgctttggtgtcttagtctcggctgccctgtaatcgaaagtatattcatcatcaaattaccaaacataaagaagcaatgatgagtctatcatctacaaaaacaatgttatgtatcccaaacctaccgattattccaaaactagtgacaagctaaggatattgtggagatgaagatgagaaagagtacgaaagctaactttgaggtttcttcttggatccaattgcgaatatgcttcacgtttcgctttagaacggaggacgctttctttgttaggcccattagcctgggctctcgtgtttttcataatgtcaagtcagcccaacaagcccaaatctttacaaaaagaaccaaggaccatgtcatccggaatatggtgatattattggattataccattggaccatttaacacaaaagcaaatatgcaacagaatatatatagatcacataacagtcagaatcttctcaaaagatatgctttcttccttttctattctagagtgttttgatgacatcggc

SEQ ID NO: 2 - DNA, 1359 bp, Artificial Sequence
  Chemically synthesized AtWRKY45 cDNA sequence
gtttgaaatttgaatccattgaaccaaaatttgaaggagttgcatatataataatataaatcagaatgatgtagccgccacacctttttgtttccacaaaactctttttctgtgatggatccgctaatgtagccatattttcaatatatatcactttctctggcatcttcgctaccgtgtacgtctctctttctctccctcccctccttggcttttttcaagttcccaccataaacgcagagggagttaagaaatggaggataggaggtgtgatgtgttgtttccatgttcatcatcggttgatcctcgcttgacagagtttcatggggtcgacaactctgctcagccgacaacatcatccgaagagaagccaaggagtaagaagaagaagaaagagagagaagcgaggtacgcgttccagacaagaagccaggttgatatactggatgatggatacaggtggaggaagtacggccaaaaagcagtcaagaacaatccattccccaggagctattataagtgcacagaagaaggatgcagagtgaagaagcaagtgcagaggcaatggggagacgaaggagtggtggtgacgacataccaaggtgttcatacacatgccgttgataaaccctctgataatttccaccacatcttgacacaaatgcacatcttccctcccttttgcttgaaggaatgattagaggaattggattgtaatatttactttcccaaaaacgttgggctcacaccatcagacctttacttttaaactagcagcaactcacatatctcaaaaatactaatccttatctttgtctttatgggacctttgaatccatctgctttggtgtcttagtctcggctgccctgtaatcgaaagtatattcatcatcaaattaccaaacataaagaagcaatgatgagtctatcatctacaaaaacaatgttatgtatcccaaacctaccgattattccaaaactagtgacaagctaaggatattgtggagatgaagatgagaaagagtacgaaagctaactttgaggtttcttcttggatccaattgcgaatatgcttcacgtttcgctttagaacggaggacgctttctttgttaggcccattagcctgggctctcgtgtttttcataatgtcaagtcagcccaacaagcccaaatctttacaaaaagaaccaaggaccatgtcatccggaatatggtgatattattggattataccattggaccatttaacacaaaagcaaatatgcaacagaatatatatagatcacataacagtcagaatcttctcaaaagatatgctttcttccttttctattctagagtgttttgatgacatcggc

SEQ ID NO: 3 - DNA, 444 bp, Artificial Sequence
  Chemically synthesized AtWRKY45 coding sequence with start and stop codon
atggaggataggaggtgtgatgtgttgtttccatgttcatcatcggttgatcctcgcttgacagagtttcatggggtcgacaactctgctcagccgacaacatcatccgaagagaagccaaggagtaagaagaagaagaaagagagagaagcgaggtacgcgttccagacaagaagccaggttgatatactggatgatggatacaggtggaggaagtacggccaaaaagcagtcaagaacaatccattccccaggagctattataagtgcacagaagaaggatgcagagtgaagaagcaagtgcagaggcaatggggagacgaaggagtggtggtgacgacataccaaggtgttcatacacatgccgttgataaaccctctgataatttccaccacatcttgacacaaatgcacatcttccctcccttttgcttgaaggaatga

SEQ ID NO: 4 - protein, 147 aa, Arabidopsis thaliana
MEDRRCDVLFPCSSSVDPRLTEFHGVDNSAQPTTSSEEKPRSKKKKKEREARYAFQTRSQVDILDDGYRWRKYGQKAVKNNPFPRSYYKCTEEGCRVKKQVQRQWGDEGVVVTTYQGVHTHAVDKPSDNFHHILTQMHIFPPFCLKE

SEQ ID NO: 5 - protein, 149 aa, Camelina sativa
MEDRYQMFFPCSSSVTKVDNSTQCGAQPTASSSSSHQNINTNEAEKPKSKMKKERESRFSFQTRSQVDILDDGYRWRKYGQKAVKNNIFPRSYYKCTQEGCRVKKQVQRLLGDEGVVVTTYQGVHTHPVDKPSDNFHHILTQMHIFPSF

SEQ ID NO: 6 - protein, 166 aa, Gossypium hirsutum
MENYQMFFPISAPSTAAQSLPLNMAPNSQAFNSFHGNSVDGFLGLKSNEDLIQKPEAKDFMKSSQKMEKKIRKPRYAFQTRSQVDILDDGYRWRKYGQKAVKNNKFPRSYYRCTHEGCKVKKQVQRLTKDESVVVTTYEGMHTHPIQKPTDNFEHILSQMQIYTPF

SEQ ID NO: 7 - protein, 223 aa, Prunus avium
MEKYQMFFPCSSSTSSANYDPMIPISATNNITTDDHHMGMGSSQVYNYFDGRDRSSNGLLGLRSSAENHVGREVLINKDHHQYLQQQYSDLTTTASANININNVVVGADQNPHEATNSGNKNKGEKKTRKPKYAFQTRSQVDILDDGYRWRKYGQKAVKNNKFPRSYYRCTYQGCNVKKQVQRLTKDEGIVVTTYEGMHTHPIEKPSDNFEHILNQMQIYTPF

SEQ ID NO: 8 - protein, 223 aa, Prunus persica
MEKYQMFFPCSSSTSSANYDPMIPISATNNITTDGHHMGMGSSQVYNYFDGRDQSSNGLLGLRSSAGNHVGREVLINKDHHQYLQQQYSDLTTTASANININNVIVGADQNPHEATNSGNKNKGEKKTRKPKYAFQTRSQVDILDDGYRWRKYGQKAVKNNKFPRSYYRCTYQGCNVKKQVQRLTKDEGIVVTTYEGMHTHPIEKPSDNFEHILNQMQIYTPF

SEQ ID NO: 9 - protein, 139 aa, Malus domestica
MSEMEASNNMIKNNFSSQGKSFGGSESGEATVRLGMKKGDQKKIRKPRYAFQTRSQVDILDDGYRWRKYGQKAVKNNKFPRSYYRCTHHGCNVKKQVQRLTKDEGVVVTTYEGMHSHPIEKSTDNFEHILSQMKIYTPF

SEQ ID NO: 10 - protein, 191 aa, Rosa chinensis
METYPTFYSSSSTTPPAAASSLSLNMVNSHPHHAYSNDQYQASNNKSNGFLGLMSEMEVSNSINSMSSISQSMKSFGEGESNTAVRAGMKKGEKKIRKPRYAFQTRSQVDILDDGYRWRKYGQKAVKNNKFPRSYYRCTHQGCNVKKQVQRLTKDEGVVVTTYEGMHSHPIEKSTDNFEHILTQMQIYTSF

SEQ ID NO: 11 - protein, 220 aa, Zea mays
MENYHMLFGTTATHAQPSSAAGTPSSYNFIATSSASGLRRDHDRGQHSGHVHAAGGSSSPSSFFVAERLSHNDDSSKDGGGGPGPAAAGSGEKEAEADDRPAAARRKGEKKERRPRFAFQTRSQVDILDDGYRWRKYGQKAVKNNNFPRSYYRCTHQGCNVKKQVQRLSRDEGVVVTTYEGTHTHPIEKSNDNFEHILTQMQIYSGMGSTFSRSSHDMFH

SEQ ID NO: 12 - protein, 195 aa, Glycine max
MENYSMLFPISNSSSYPISTSGVGSSQIGYNGQSSNAFLGLRPSNELLGSDDHDNGGEGGGDGDGNMLMSQISGGSNTNVSDELGGSGNSNNKKKGEKKVKKPRYAFQTRSQVDILDDGYRWRKYGQKAVKNNKFPRSYYRCTHQGCNVKKQVQRLTKDEGVVVTTYEGVHTHPIEKTTDNFEHILSQMKIYTPF

SEQ ID NO: 13 - protein, 242 aa, Oryza sativa
MENFPILFATQPTSSSTSSSYHFMSSSSGSHDHRHHHGLQAGGNGGGGGGSLSHGLFMGSSSSSIRMEELSNSKQAGDVVVDGGATRSPHGGDGDGAAGDDGGDAQAAAAGGRKKGEKKERRPRFAFQTRSQVDILDDGYRWRKYGQKAVKNNKFPRSYYRCTHQGCNVKKQVQRLSRDETVVVTTYEGTHTHPIEKSNDNFEHILTQMHIYSGLTPSSAAHASSSSPLFPSAAAAASHMFQ

SEQ ID NO: 14 - protein, 131 aa, Solanum lycopersicum
MDINDVVVGSVSERMQNDHRNLLSVKNKKIKKPRFAFQTKSQVDILDDGYRWRKYGQKAVKNNNYPRSYYRCTHEGCNVKKQVQRLSKDETVVVTTYEGMHTHPIQKPNDNFEQILHQMHIFPNPPCHLIN

SEQ ID NO: 15 - protein, 172 aa, Solanum tuberosum
MENYATIFPSASSSSSHHDEYISLMNSKSSISDDAKEELLFQGKNKAGFLGLMASMETPRDIITKKDEVIKSCKKKIKKPRYAFQTRSQVDILDDGYRWRKYGQKAVKNNKFPRSYYRCTHQGCNVKKQVQRLSKDEEVVVTTYEGMHSHPIDKSTDNFEHILSQMQIYTSF

SEQ ID NO: 16 - protein, 145 aa, Arabidopsis thaliana
MEGYDNGSLYAPFLSLKSHSKPELHQGEEESSKVRSEGCSKSVESSKKKGKKQRYAFQTRSQVDILDDGYRWRKYGQKAVKNNKFPRSYYRCTYGGCNVKKQVQRLTVDQEVVVTTYEGVHSHPIEKSTENFEHILTQMQIYSSF

SEQ ID NO: 17 - DNA, 21 bp, Artificial Sequence
  Chemically synthesized AtWRKY45 forward primer
atggaggataggaggtgtgat

SEQ ID NO: 18 - DNA, 22 bp, Artificial Sequence
  Chemically synthesized AtWRKY45 reverse primer
tcattccttcaagcaaaaggga

SEQ ID NO: 19 - DNA, 21 bp, Artificial Sequence
  Chemically synthesized 35S promoter specific forward primer
ggagaggacctcgactctaga